(19) United States
(12) Patent Application Publication
(10) Pub. No.: US 2017/0224351 A1
(43) Pub. Date: Aug. 10, 2017

(54) ENDOSCOPIC MULTI-CLIP DISPENSING APPARATUS

(71) Applicant: Mubashir H. Khan, Springfield, MO (US)

(72) Inventor: Mubashir H. Khan, Springfield, MO (US)

(21) Appl. No.: 15/401,526

(22) Filed: Jan. 9, 2017

Related U.S. Application Data
(63) Continuation-in-part of application No. 14/276,513, filed on May 13, 2014, now abandoned.
(60) Provisional application No. 61/961,842, filed on Oct. 24, 2013.
(60) Provisional application No. 61/855,313, filed on May 14, 2013.
(60) Provisional application No. 61/957,306, filed on Jun. 29, 2013.

(51) Int. Cl. A61B 17/128; A61B 1/005; A61B 17/29; A61B 1/018; A61B 17/00; A61B 1/00; A61B 1/06

(52) U.S. Cl. CPC A61B 17/1285; A61B 1/00195; A61B 1/0057; A61B 1/0669; A61B 1/018; A61B 17/00234; A61B 17/29; A61B 2017/0034; A61B 2017/00323

(57) ABSTRACT

An endoscopic multi-clip dispensing apparatus has elongated inner and outer conduits sleeved together, wherein the inner conduit reciprocates between extension and retraction. A plunger rod pushes a line of clips through the core of the inner conduit at an indexed rate of one clip at a time. Each C-shaped clip has a pair of elongated jaws mated together at a hinge end. The clips are loaded into the inner conduit in head to tail fashion, with the jaw ends of a trailing clip pushing the hinge end of a leading clip. There further is a pair of wedge-shaped flared structures proximate the dispensing end of the conduits, for prying open the jaws of the lead clip during the progression of it being dispensed. Ultimately, after the lead clip passes clear of the flared structures, its jaws are free to snap shut and ‘bite’ into a target anatomy.

### CROSS-REFERENCE TO PROVISIONAL APPLICATION(S)

This application is a continuation-in-part of U.S. patent application Ser. No. 14/276,513, filed May 13, 2014; which claims the benefit of U.S. Provisional Application No. 61/961,842, filed Oct. 24, 2013; U.S. Provisional Application No. 61/957,306, accorded filing date of Jun. 29, 2013; and, U.S. Provisional Application No. 61/855,313, accorded filing date of May 14, 2013. The foregoing disclosures are incorporated herein by this reference thereto.

### BACKGROUND AND SUMMARY OF THE INVENTION

The invention relates to surgery and, more particularly, to endoscopic multi-clip dispensing apparatus.

It is an object of the invention to line up a series of surgery clips in accordance with the invention in a line where the head of each clip is pushing into the tail of each preceding clip, until the leadmost clip.

It is another object of the invention to provide a dispensing apparatus in accordance with the invention for dispensing, at first, the leadmost clip, and then serially each succeeding clip thereafter when and where chosen by the operator (eg., surgeon).

A number of additional features and objects will be apparent in connection with the following discussion of the preferred embodiments and examples with reference to the drawings.

### BRIEF DESCRIPTION OF THE DRAWINGS

There are shown in the drawings certain exemplary embodiments of the invention as presently preferred. It should be understood that the invention is not limited to the embodiments disclosed as examples, and is capable of variation within the scope of the skills of a person having ordinary skill in the art to which the invention pertains. In the drawings,

FIG. 1A is a perspective view of a first embodiment of endoscopic multi-clip dispensing apparatus in accordance with the invention for dispensing a first embodiment of C-shaped surgery clips in accordance with the invention, as snaked through an operating channel of an endoscope, wherein proximate the terminal end of the endoscope, only the outline of the endoscope as well as the control wires for flexing (ie., curling) the endoscope are shown in dashed lines;

FIG. 1B is a perspective view of the endoscope of FIG. 1A;

FIG. 1C is an enlarged scale section view taken along the line 1C-1C in FIG. 1B, with only the outline of the endoscope shown in dashed lines;

FIG. 2 is an enlarged scale perspective view of one such surgery clip, as with its jaws forced open;

FIG. 3 is a perspective view comparable to FIG. 2 except showing the clip in the state of normally snapped shut;

FIG. 3A is a sectional view taken along line IIIA-IIIA in FIG. 3;

FIG. 3B is a perspective view comparable to FIG. 3 except showing the composite construction of the clip wherein the spring portion is shown in solid lines (and is preferably a spring material like a spring steel) and the remaining portions shown in dashed lines might comprise a different material, such as non-spring stainless steel;

FIG. 4 is an enlarged scale perspective view of the dispensing end of said endoscopic multi-clip dispensing apparatus, wherein the outline of the endoscope as well as the control wires for flexing (ie., curling) the endoscope are shown in dashed lines;

FIG. 5 is a perspective view comparable to FIG. 4 except showing a lead clip being advanced through the dispensing end of endoscopic multi-clip dispensing apparatus and out through the very end margin of the dispensing end's tubewall, wherein wedge-shaped, jaw-spreading flared ends of a pair of actuator arms are also extending through a driven extension stroke to project out the end of the dispenser;

FIG. 6 is a perspective view comparable to FIG. 5 except showing further advance of the process of dispensing of the lead clip;

FIG. 7 is a perspective view comparable to FIG. 6 except showing the lead clip being pushed further forward such that the wedge-shaped flared ends of the actuator arms have spread open wide the jaws of the clips (note, this is accomplished by the left and right flanges riding over the left and right flared ends);

FIG. 8 is an enlarged scale sectional view taken along the line VIII-VIII from FIG. 4;

FIG. 9 is an enlarged scale end elevational view taken in the direction of arrows IX-IX from FIG. 8;

FIG. 10 is an enlarged scale sectional view taken along the line X-X from FIG. 8;

FIG. 11 is an enlarged scale sectional view taken along the line XI-XI from FIG. 8;

FIG. 12 is an enlarged scale sectional view taken along the line XII-XII from FIG. 8;

FIG. 13 is a side elevational view, partly in section, take along line from FIG. 8;

FIG. 14 is a side elevational view, partly in section, comparable to FIG. 13 except showing the coordinated extension of one flared end of one of the pair of actuator arms and projection of the lead clip out the dispensing end;

FIG. 15 is a side elevational view, partly in section, comparable to FIG. 14 except showing the end of travel for the extension of the one flared end of the one of the pair of actuator, arms, in contrast to, the continued travel of the lead clip out the dispensing end;

FIG. 16 is a side elevational view, partly in section, comparable to FIG. 15 except showing the continued travel of the lead clip out of the dispensing end relative to the now stationary one flared end of the one of the pair of actuating arms, wherein the lead clip's flanges have engaged the wedge-shaped flared end and have been wedged apart such the jaws of the lead clip are spread wide open;

FIG. 17 is a side elevational view, partly in section, comparable to FIG. 16 except showing not only the clearance of the lead clip out of the dispensing end but also the clearance of the flanges past the one flared end of the one of the pair of actuating arms, whereby the jaws of the clip automatically start to spring shut;

FIG. 18 is a side elevational view, partly in section, comparable to FIG. 13 except omitting the illustration of any clip; and

FIG. 19 is an enlarged scale sectional view taken along line XIX-XIX from FIG. 1A and showing the abutting end of the urging follower that pushes on the last clip of the indefinitely long series of the first embodiment clips in accordance with the invention that would be loaded into the first embodiment endoscopic multi-clip dispensing apparatus in accordance with the invention;

FIG. 20 is a perspective view of another embodiment of endoscopic multi-clip dispensing apparatus in accordance with the invention for dispensing another embodiment of C-shaped surgery clips also in accordance with the invention;

FIG. 21 is an enlarged scale perspective view of one such surgery clip, as with its jaws in the state of normally snapped shut;

FIG. 22 is a perspective view comparable to FIG. 21 except showing the clip with its jaws in the state of forced open;

FIG. 23 is a top plan view of FIGS. 21;

FIG. 24 is a side elevational view of FIG. 23, wherein dashed lines at the tail end show that varying the thickness of the tail end varies the clamping power of said clip;

FIG. 25A is an enlarged scale perspective view of the dispensing end of the dispensing apparatus of FIG. 20 and showing aspects of the dispensing arms of said dispensing apparatus, as snaked through an operating channel of an endoscope, wherein only the outline of the endoscope as well as the control wires for flexing (ie., curling) the endoscope are shown in dashed lines;

FIG. 25B is a perspective view of the terminal end of the endoscope of FIG. 25A;

FIG. 25C is an enlarged scale section view taken along the line XXVC-XXVC in FIG. 25B, with only the outline of the endoscope shown in dashed lines;

FIG. 26 is a sectional view taken along line XXVI-XXVI in FIG. 25A, except with the clip(s) removed from the view;

FIG. 27 is a sectional view taken along line XXVII-XXVII in FIG. 26;

FIG. 28 is a sectional view comparable to FIG. 26, except with the lead clip entering the ejection station for the dispensing end of said dispensing apparatus in accordance with the invention;

FIG. 29 is an enlarged scale end elevational view taken in the direction of arrows XXIX-XXIX from FIG. 28 and to show better show the ovate-shaped, jaw-spreading flared-ends of the actuator arms of said dispensing apparatus in accordance with the invention;

FIG. 30 is an enlarged scale sectional view taken in the direction of the arrows XXX-XXX from FIG. 28;

FIG. 31 is an enlarged scale sectional view taken along the line XXXI-XXXI from FIG. 28;

FIG. 32 is an enlarged scale sectional view taken along line XXXII-XXXII in FIG. 35 and comparable to FIG. 31 except wherein the lead clip has been advanced down the hollow passage of the flexible conduit of said dispensing apparatus in accordance with the invention (ie., by means of for example and without limitation being pushed from behind), and wherein the jaws of the lead clip have opened slightly;

FIG. 33 is an enlarged scale sectional view taken along line XXXIII-XXXIII in FIG. 35 and comparable to FIG. 32 except showing the plunger rod which serves as the pressure-urging follower that follows the trailing clip of the series of clips loaded head to tail in the hollow passage of the flexible conduit of said dispensing apparatus in accordance with the invention, wherein said plunger rod advances the whole series of clips forward in unison by abutting and applying pressure against the tail end of the trailing clip (see, eg., FIG. 35);

FIG. 34 is a perspective view comparable to FIG. 25 except showing the ejection process of the lead clip advancing through the dispensing end of the conduit such that the lead clip has moved relatively forward and that the actuator arms have thrust relative forward such that the ovate-shaped, jaw-spreading flared-ends thereof extend outside the dispensing end of said dispensing apparatus in accordance with the invention;

FIG. 35 is a perspective view comparable to FIG. 34 except showing the ejection process of the lead clip advancing through the dispensing end of the conduit such that the lead clip has moved relatively forward and that the ovate-shaped, jaw-spreading flared-ends of the actuator arms have begun to urge the jaws of the clip relatively apart;

FIG. 36 is a sectional view partly comparable to FIG. 27 except showing the arms partly advanced forward and showing the relative position of the lead clip;

FIG. 37 is a sectional view partly comparable to FIG. 36 except showing further advance of the arms and lead clip;

FIG. 38 is a sectional view partly comparable to FIG. 37 except showing further advance lead clip only, wherein the arms advanced to their further extension point in FIG. 37, and here in FIG. 38 the flared ends of the arms are in the middle of being spread apart from a normal position of flexed inward toward each other (see, eg., FIGS. 36 or 37);

FIG. 39 is a sectional view partly comparable to FIG. 38 except showing further advance of the lead clip wherein, again, the arms do not advance any further, but, the flared ends do spread apart ever so slightly more;

FIG. 40 is a sectional view partly comparable to FIG. 28 except corresponding to the disposition of things in FIG. 36;

FIG. 41 is a sectional view partly comparable to FIG. 40 except corresponding to the disposition of things in FIG. 37;

FIG. 42 is a sectional view partly comparable to FIG. 41 except corresponding to the disposition of things in FIG. 38;

FIG. 43 is a sectional view partly comparable to FIG. 42 except corresponding to the disposition of things in FIG. 39;

FIG. 44 is a sectional view partly comparable to FIG. 43 except showing further advance of the lead clip, and, the beginning of the forcing apart the jaws thereof; and

FIG. 45 is a sectional view partly comparable to FIG. 43 except showing still further advance of the lead clip, and, the maximum extent of the forcing apart the jaws of the clip, after which said lead clip has been ejected clear of said dispensing apparatus and is free to clamp (eg., ‘bite’) on the target tissue which the user (eg., surgeon) has presumably taken aim upon (target tissue not shown);

FIG. 46 is a perspective view of a further embodiment of endoscopic multi-clip dispensing apparatus in accordance with the invention for dispensing a further embodiment of endoscopic surgery clips also in accordance with the invention;

FIG. 47 is an enlarged scale perspective view of one such surgery clip, as with its jaws spread open in state of full extension about 180° open wide;

FIG. 48 is a perspective view comparable to FIG. 47 except showing the surgery clip with its jaws in the process of clamping shut, but still about 30° apart;

FIG. 49 is an exploded perspective view of the clip, showing that its two jaws are resiliently biased to a shut (eg., clamping) position therefor by a torsion spring;

FIG. 50A is a perspective view of a series of such clips loaded head to tail in the hollow passage of the flexible conduit of said further embodiment of said dispensing apparatus in accordance with the invention, wherein a plunger rod advances the whole series of clips forward in unison by abutting and applying pressure against the tail end of the trailing clip (see, eg., FIG. 35), and all as snaked through an operating channel of an endoscope, wherein only the outline of the endoscope as well as the control wires for flexing (ie., curling) the endoscope are shown in dashed lines;

FIG. 50B is a perspective view of the terminal end of the endoscope of FIG. 50A;

FIG. 50C is an enlarged scale section view taken along the line LC-LC in FIG. 50B, with only the outline of the endoscope shown in dashed lines;

FIG. 51 is a side elevational view of FIG. 47;

FIG. 52 is a side elevational view of FIG. 48;

FIG. 53 is a top plan view of FIG. 51;

FIG. 54 is a bottom plan view of FIG. 51;

FIG. 55 is a side elevational view of the opposite side of FIG. 51;

FIG. 56 is a left end elevational view of FIG. 51;

FIG. 57 is a right end elevational view of FIG. 51;

FIG. 58 is a sectional view taken along line LVIII-LVIII in FIG. 51;

FIG. 59 is a sectional view taken along line LIX-LIX in FIG. 51;

FIG. 60 is an elevational view, partly in section, of the dispensing end of FIG. 46 and/or somewhat of FIG. 50;

FIG. 61 is a section view taken along line LXI-LXI in FIG. 60;

FIG. 62 is an elevational view, partly in section, and comparable to FIG. 60 except showing the advance of the lead clip out of the dispensing end of the conduit of said dispensing apparatus;

FIG. 63 is an elevational view, partly in section, and comparable to FIGS. 60 and 62 except showing the ejection of the lead clip out of the dispensing end of the conduit of said dispensing apparatus, wherein this view presupposes that the operator (eg., surgeon) of this dispensing apparatus has hooked the lead hook-end of the clip on some target anatomy (not shown), which is why the lead hook-end of the clip remains axially aligned with the core of the conduit of the dispensing apparatus and it is the trailing hook-end of the clip which swings around to ‘bite into’ (eg., form a clamp with) the lead hook-end;

FIG. 64 is an exploded perspective view comparable to FIG. 49, except of still another embodiment of an endoscopic surgery clip in accordance with the invention, which like the clip of FIG. 49 has two jaws which are torsionally-biased toward each other, and which can be spread 180° apart, except wherein the torsion applicator is bar of resilient material (which is shown suspended in isolation between the two jaws of the clip);

FIG. 65 is an enlarged-scale side elevational view comparable to FIG. 50, except of the clip of FIG. 64, and of more particularly the hinge portion thereof;

FIG. 66 is a sectional view taken along line LXVI-LXVI in FIG. 65, wherein the torsional resilient bar has been twisted into at least one full coil, whereby the uncoiling thereof into half a coil swings the jaws from wide open at 180° apart to shut at about 0° apart;

FIG. 67 is a perspective view comparable to FIG. 47, except of yet another embodiment of a clip in accordance with the invention for dispensing out of a multi-clip dispensing apparatus in accordance with the invention wherein the clips are loaded in a line in a core of an axially-elongated conduit of the dispensing apparatus, in a head to tail line up;

FIG. 68 is a side elevational view thereof, with a leading hook-end for a leading jaw to the left in the view and a trailing hook-end for a trailing jaw shown to the right in the view;

FIG. 69 is a side elevational view comparable to FIG. 68 except showing the jaws of the clip flexed open beyond a mere 180° apart, and, instead to nearly 225° apart, wherein this view presupposes that the leading jaw has cleared the dispensing end of the dispensing apparatus and the operator (eg., surgeon) of the dispensing apparatus has hooked the lead hook-end of the clip on some target anatomy (not shown), which is why the lead hook-end of the clip is bent upward of axis of the core of the conduit of the dispensing apparatus (as the trailing hook-end, which is still confined therein, remains axially-aligned with such core of the conduit of the dispensing apparatus);

FIG. 70 is a side elevational view comparable to FIG. 69 except showing the progression of the trailing jaw after it has cleared the dispensing end of the dispensing apparatus, ie., the jaws snap shut and “bite” into (eg., apply a clamping force on) the target tissues aimed at by the operator of the dispensing apparatus (eg., a surgeon); around to ‘bite into’ (eg., form a clamp with) the lead hook-end;

FIG. 71 is a side elevational view comparable to FIG. 68, except including the illustration of the dispensing end of the elongated conduit of the dispensing apparatus;

FIG. 72 is a side elevational view comparable to FIG. 69, except including the illustration of the dispensing end of the elongated conduit of the dispensing apparatus;

FIG. 73 is a side elevational view comparable to FIG. 70, except including the illustration of the dispensing end of the elongated conduit of the dispensing apparatus; and

FIG. 74 is an enlarged-scale side elevational view comparable to FIG. 69, and focusing in on the hinge-portion of said clip;

FIG. 75 is a perspective view comparable to any of FIG. 7 or 35 except of a different embodiment of a clip and dispensing apparatus in accordance with the invention, wherein unlike FIGS. 1-45 in which the clips have just a pair of jaws, the clip in this view has three jaws (ie., it is more of a claw) and the dispensing apparatus is arranged with three arms formed with flared ends to manage the opening of the jaws/claws of this clip, however, like the clips of FIGS. 1-45 the clips of this view does travel through the inner tubular conduit of the dispensing apparatus with the jaws/claws facing forwardly; and

FIG. 76 is a section view taken along line LXXVI-LXXVI in FIGS. 75.

### DETAILED DESCRIPTION OF THE PREFERRED EMBODIMENTS

The drawing figures show various embodiments of a series of surgery clips 100, 102, 104, 106 and 108 in accordance with the invention which are formed in a line in a tubular conduit 130 of endoscopic multi-clip dispensing apparatus 110, 112, 114 and/or 118 in accordance with the invention.

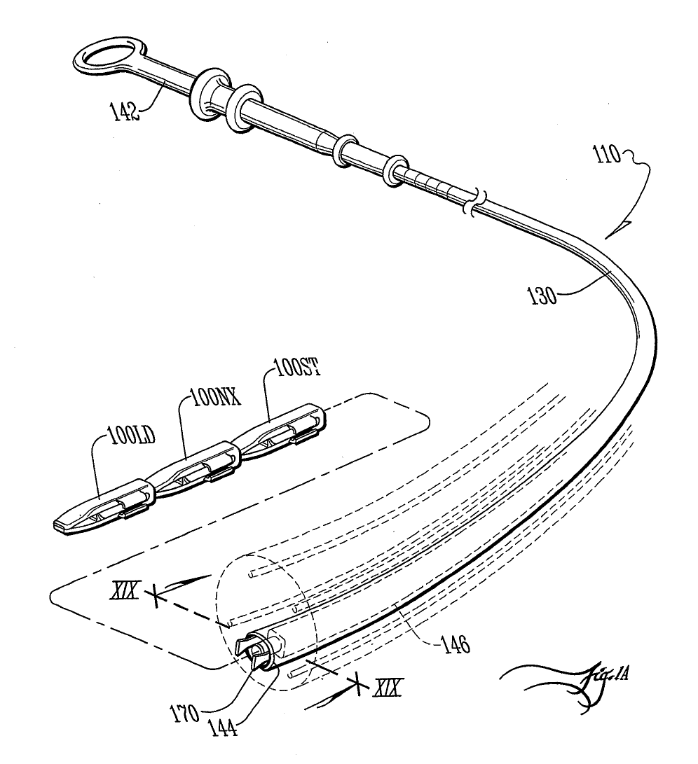

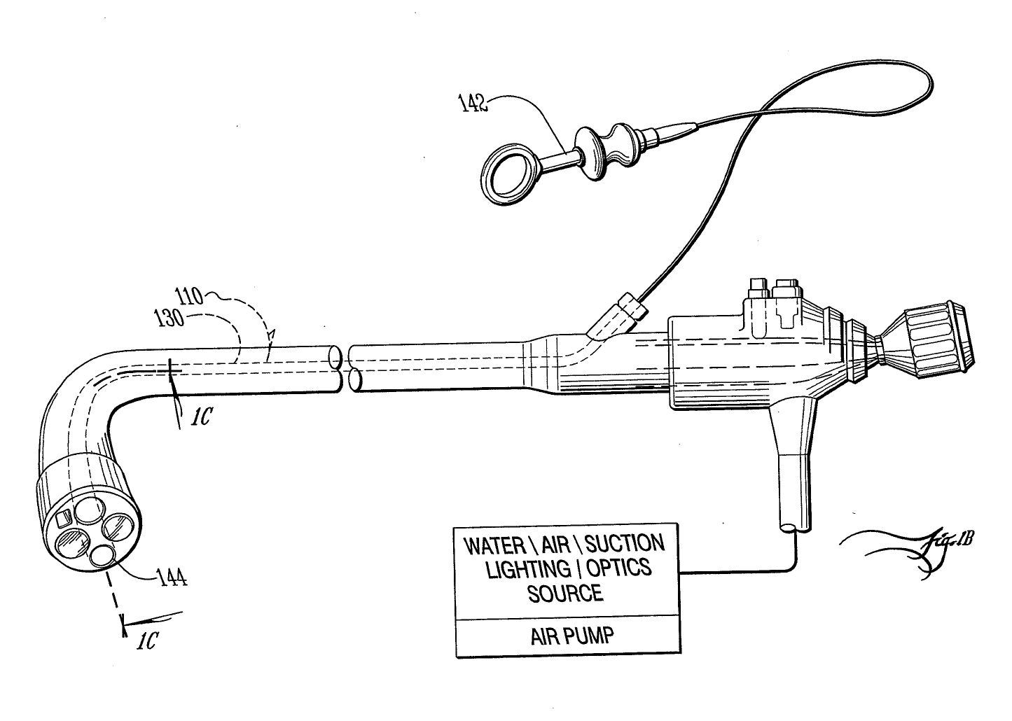

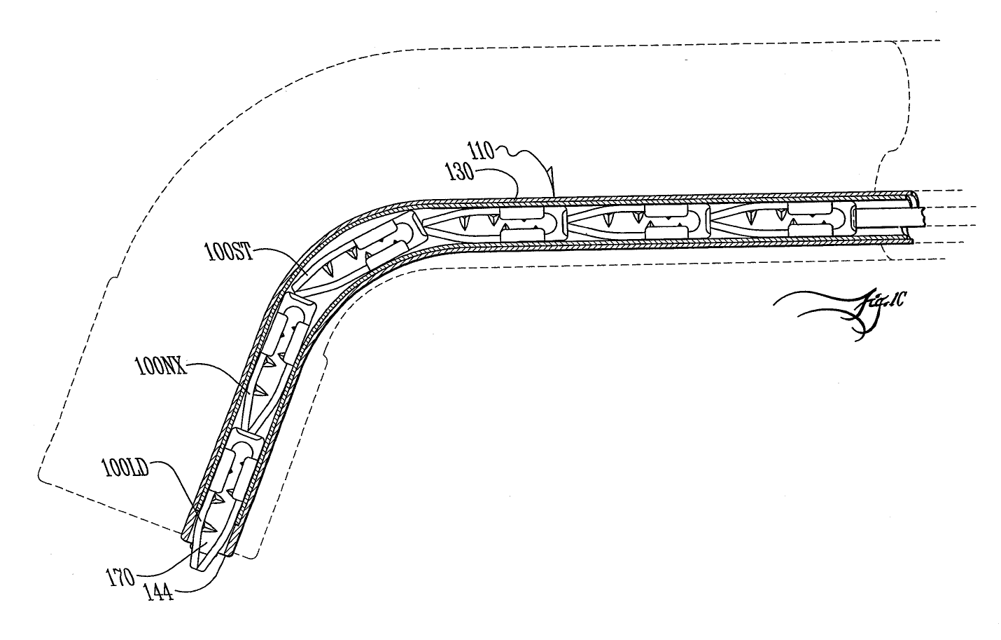

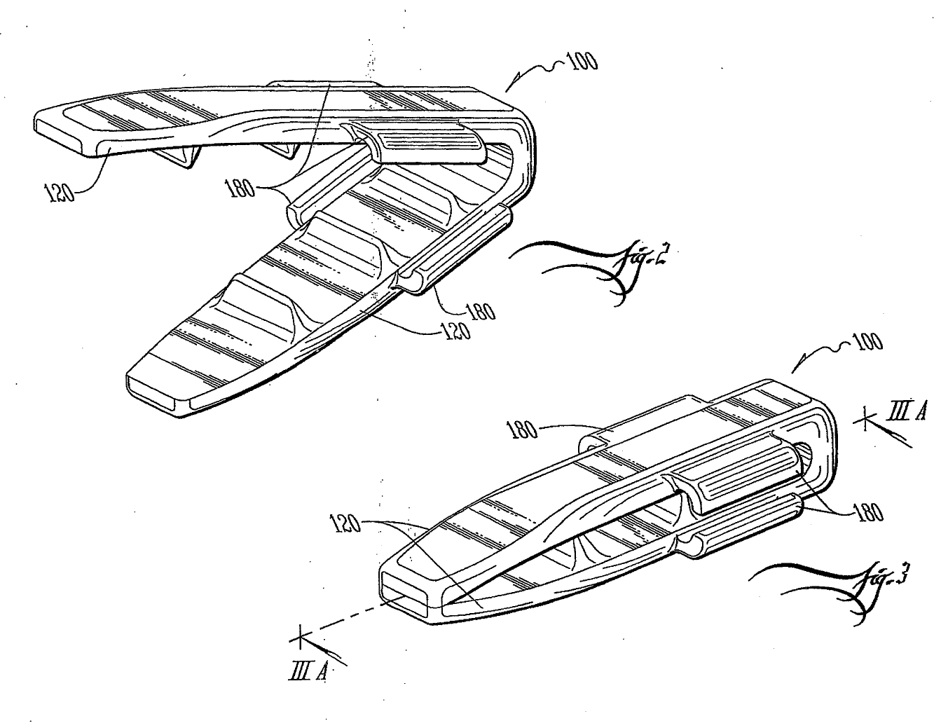

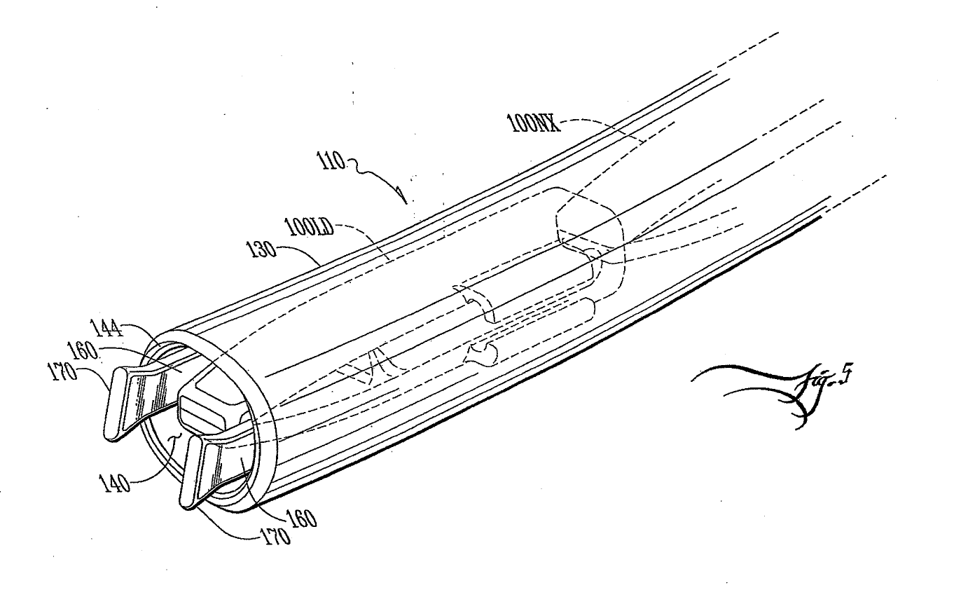

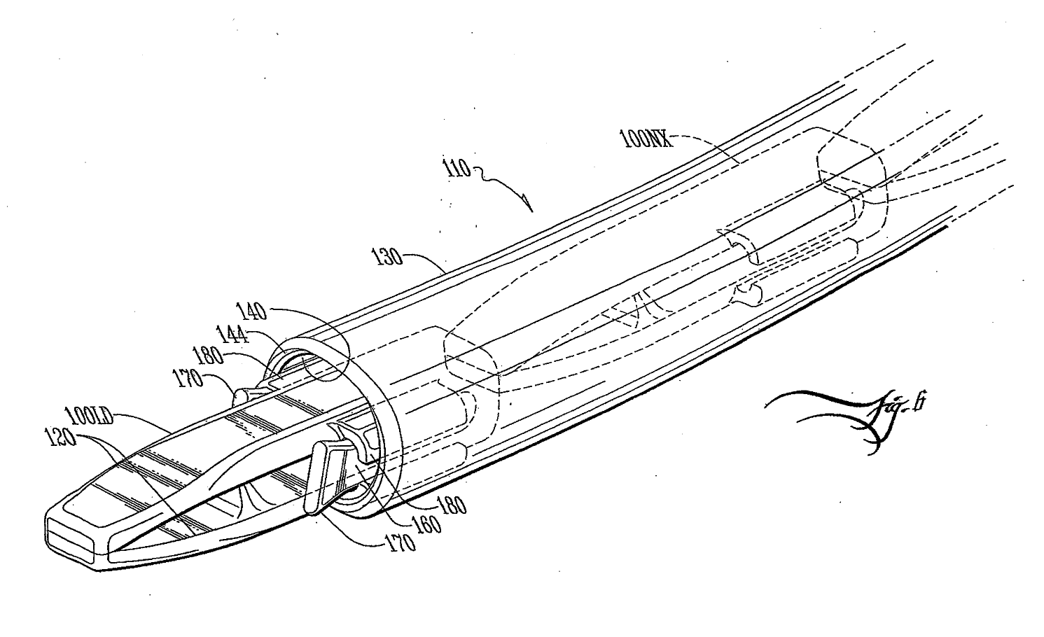

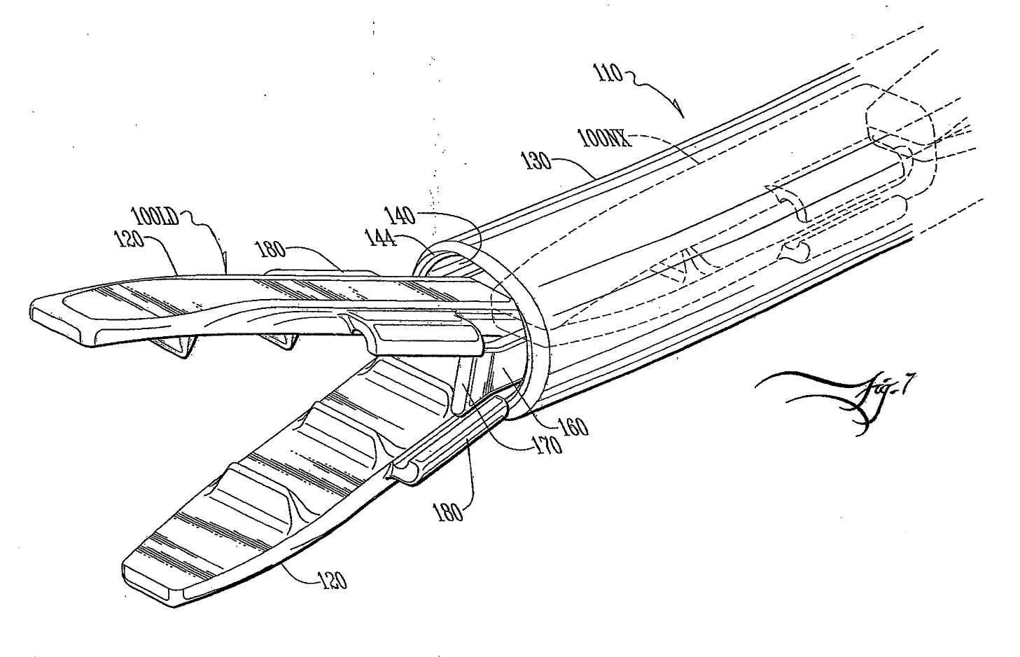

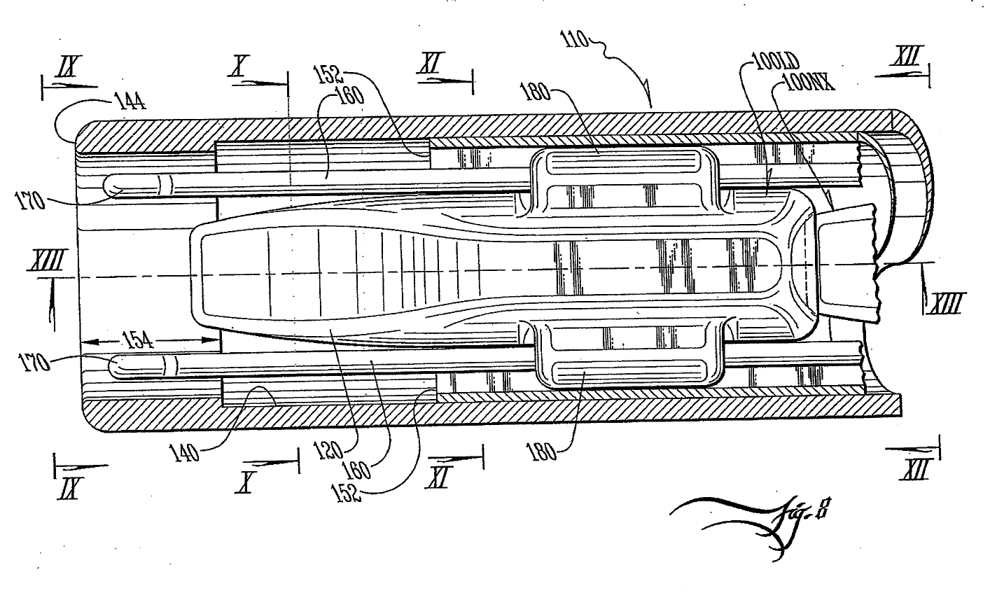

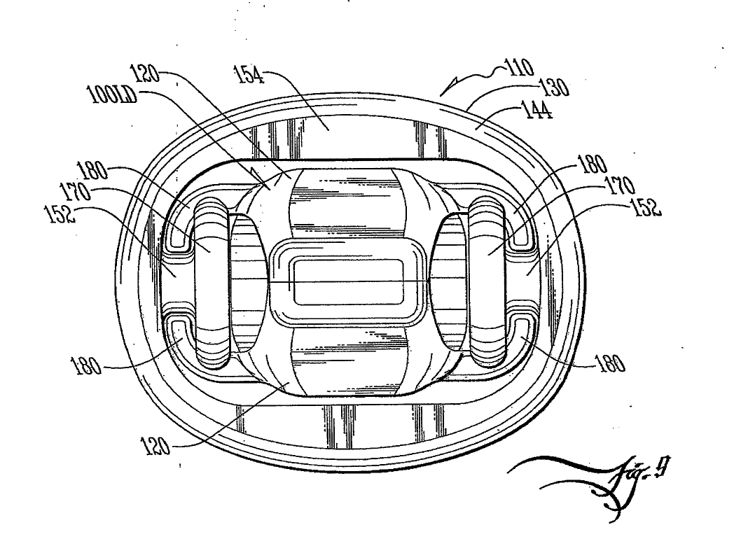

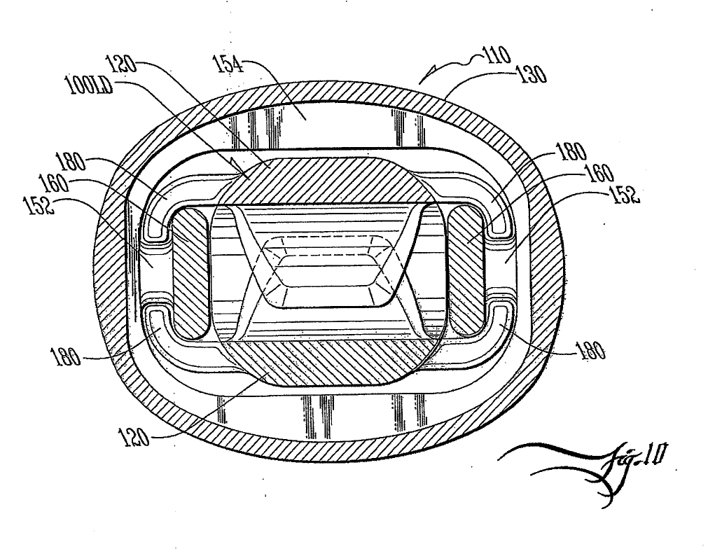

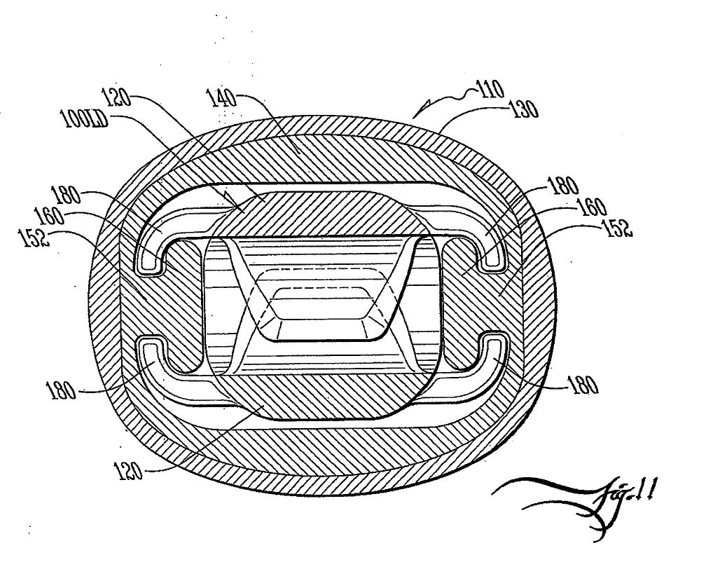

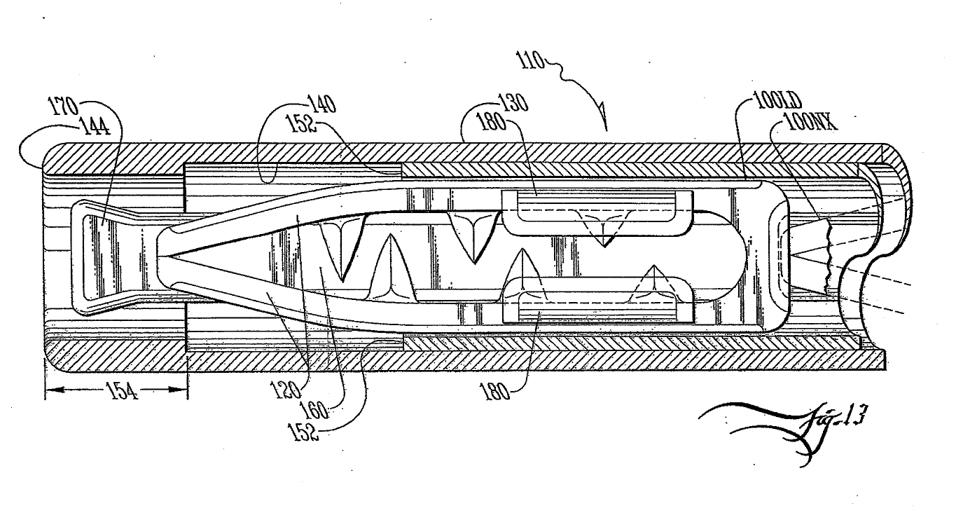

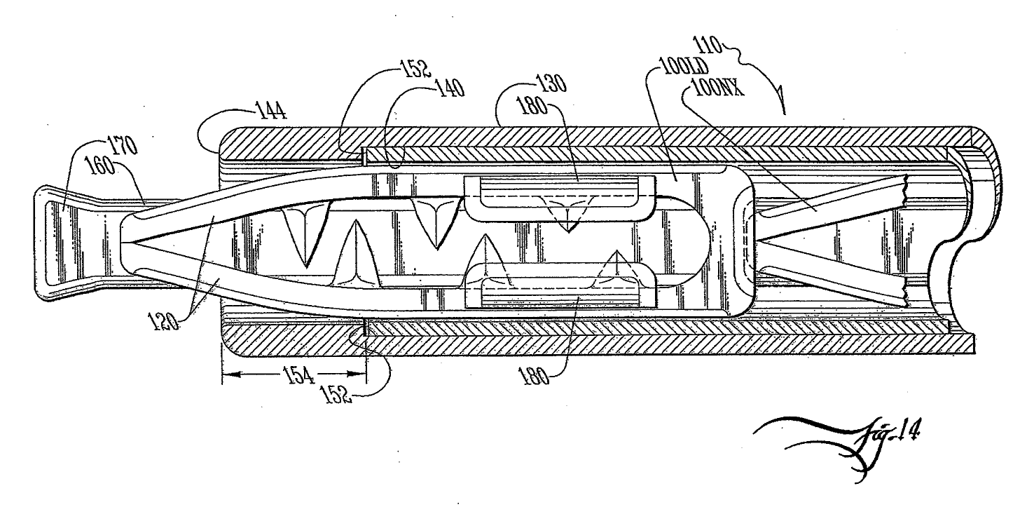

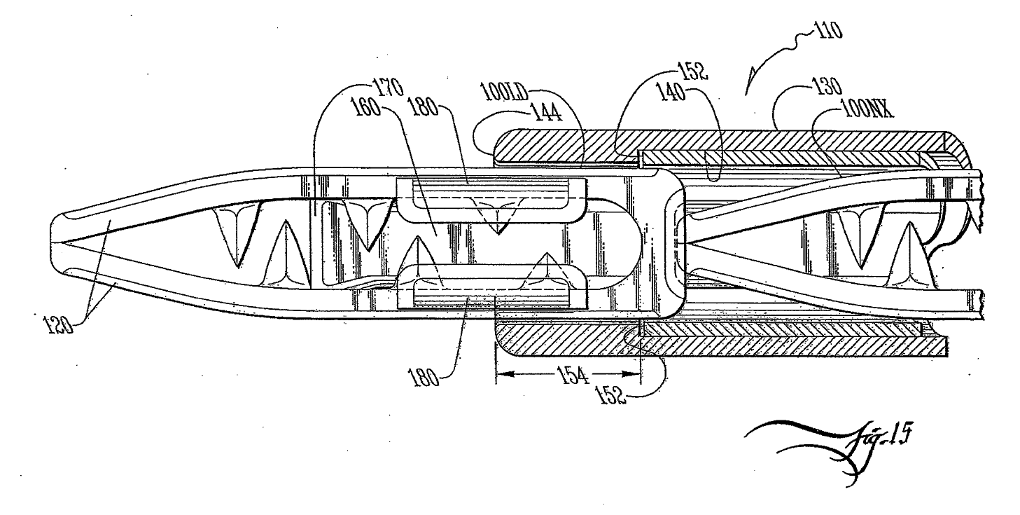

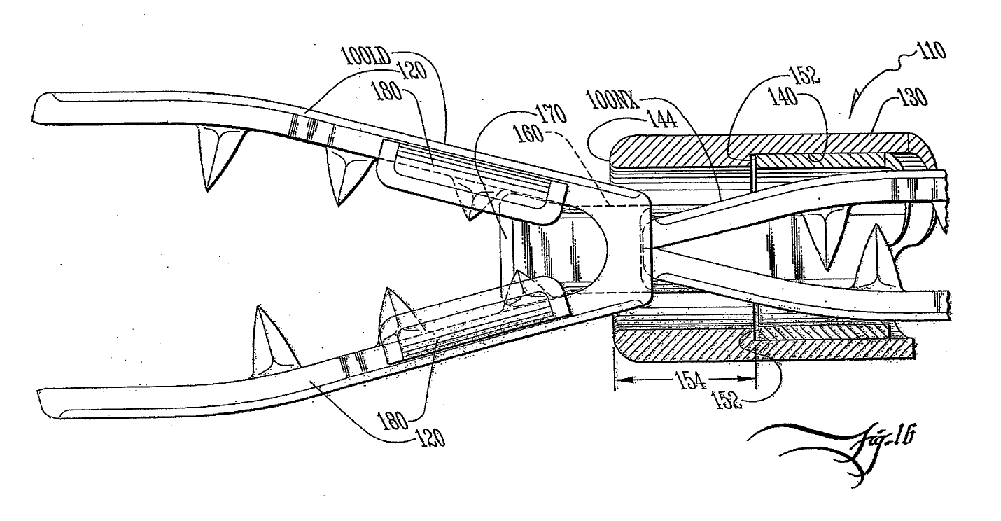

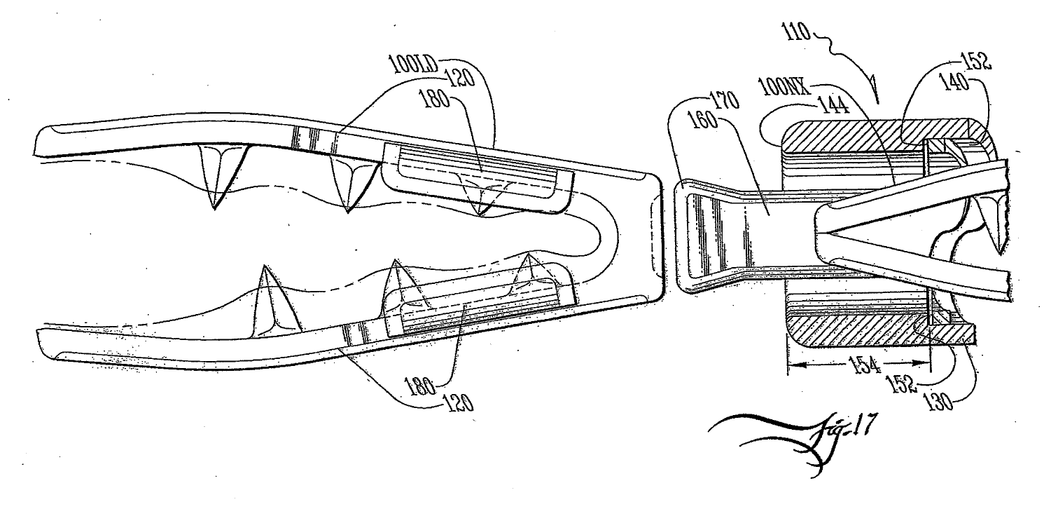

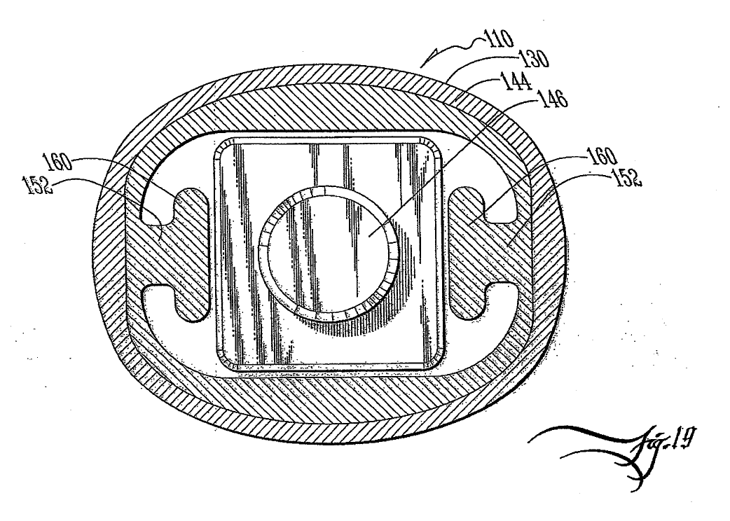

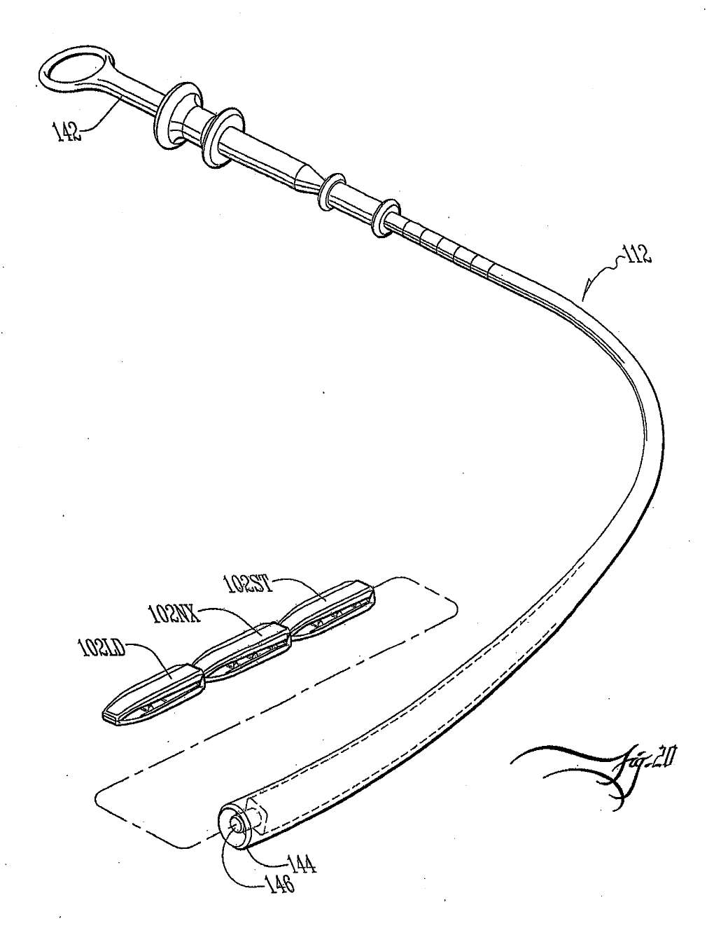

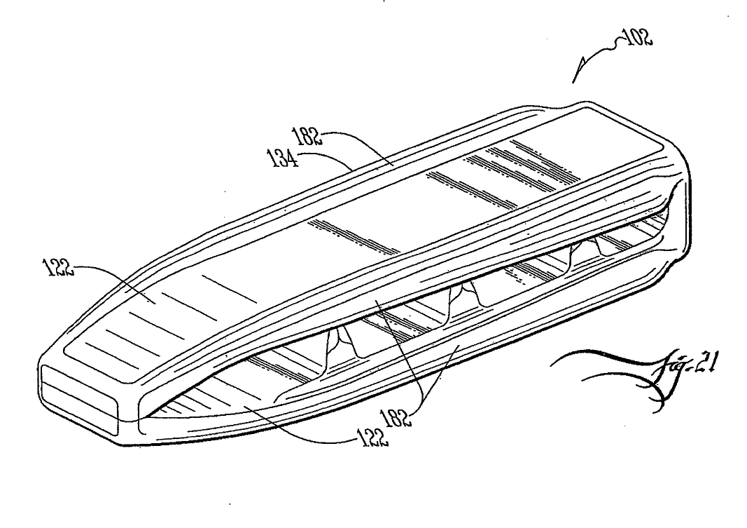

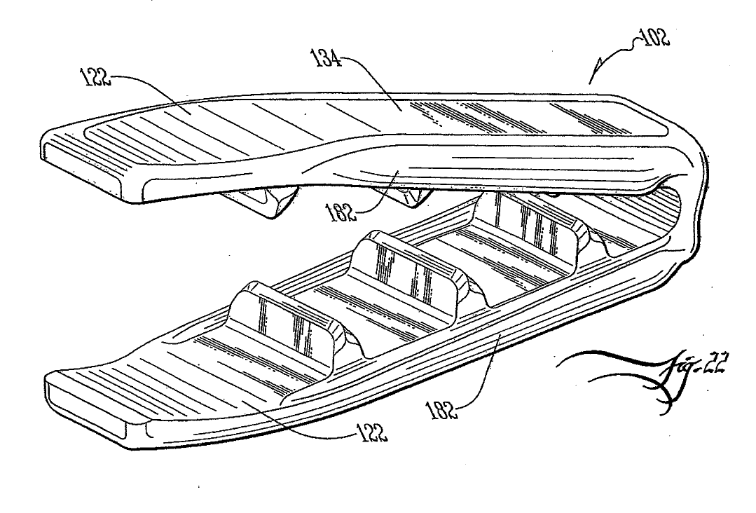

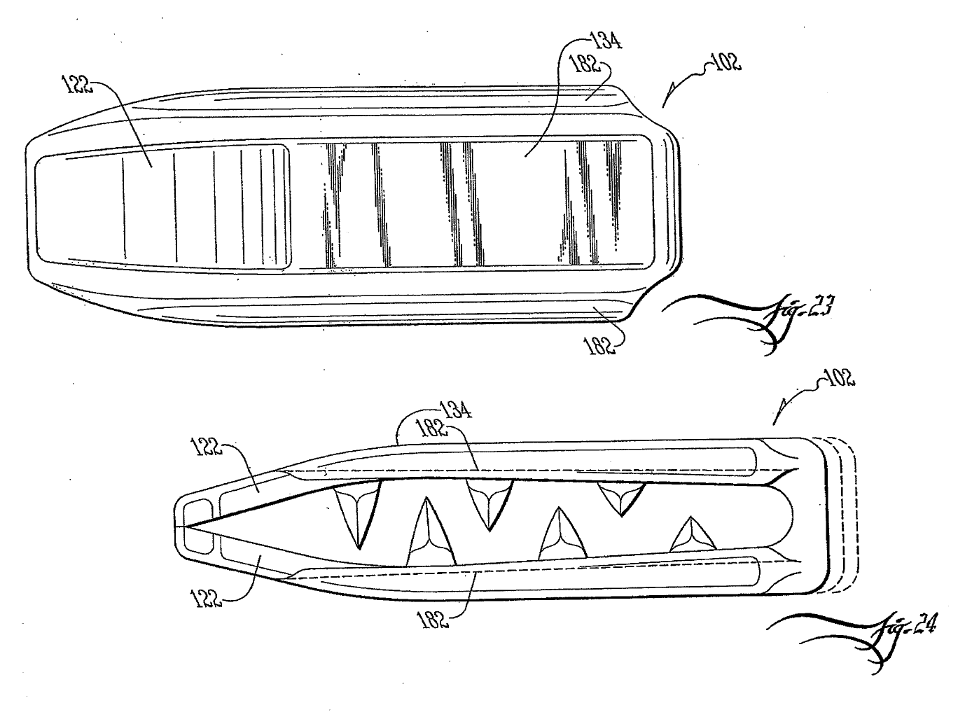

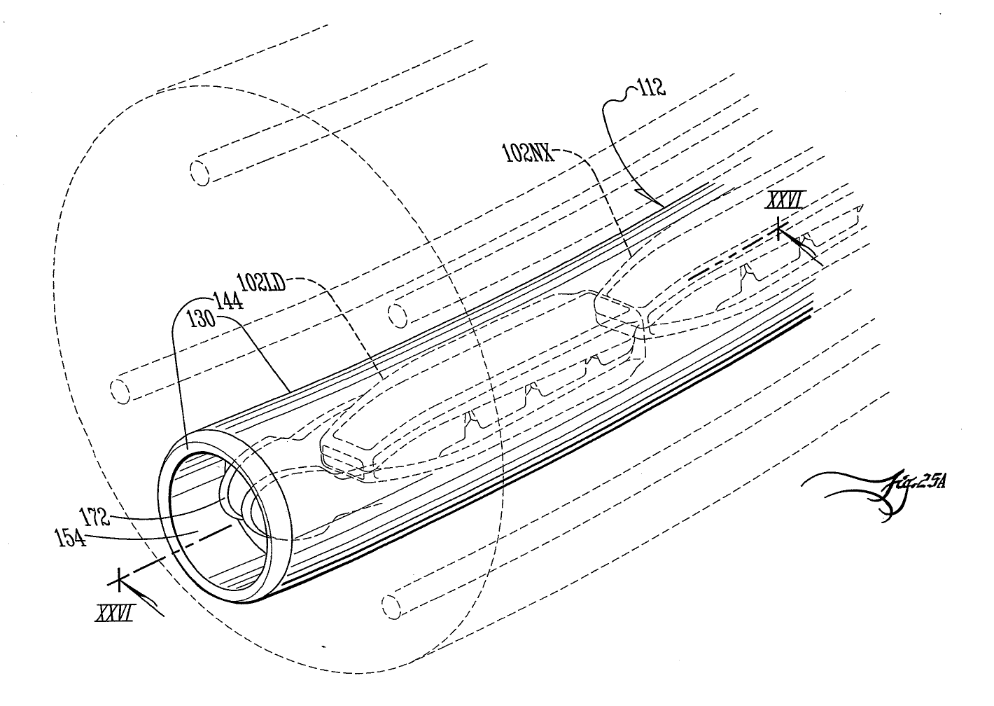

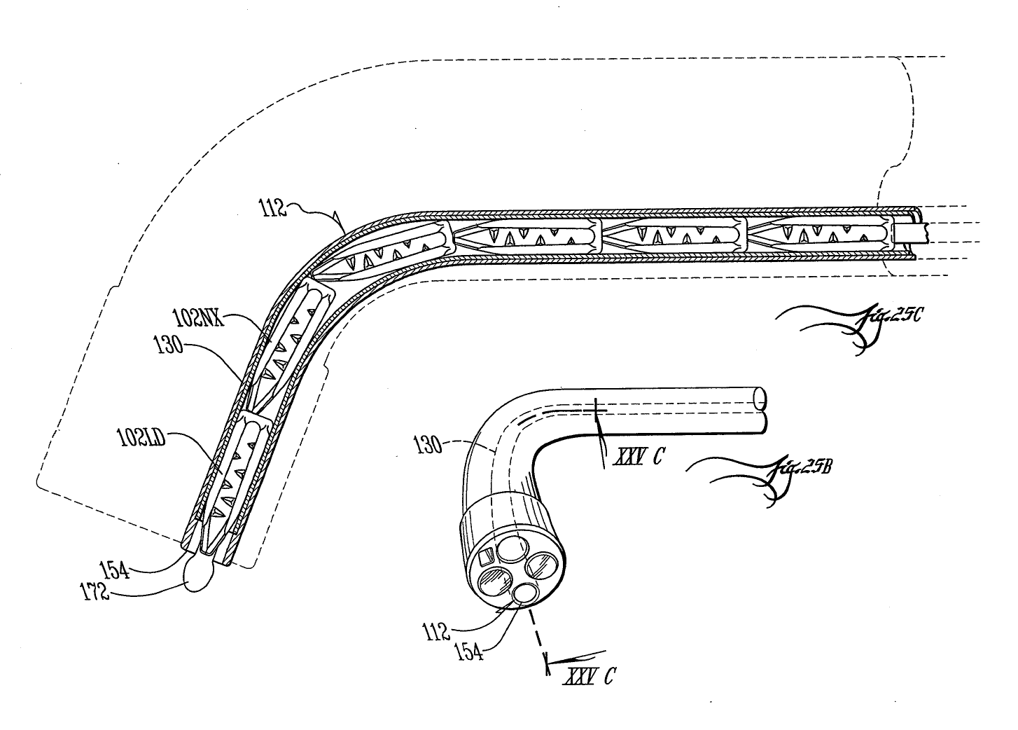

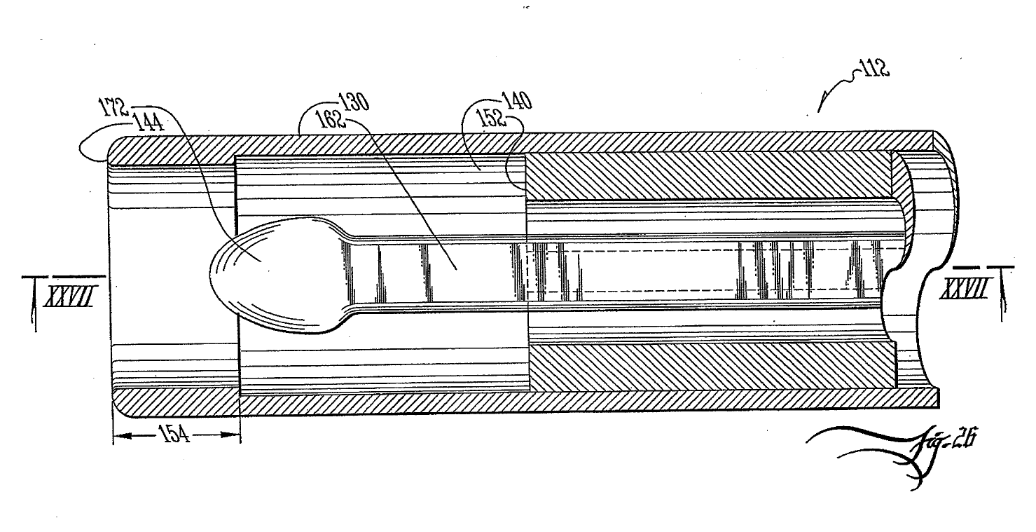

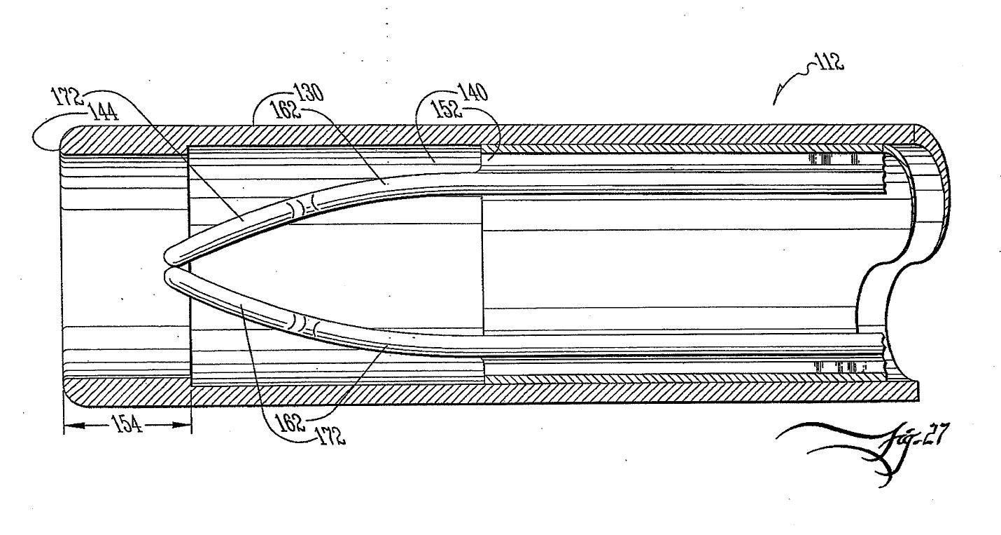

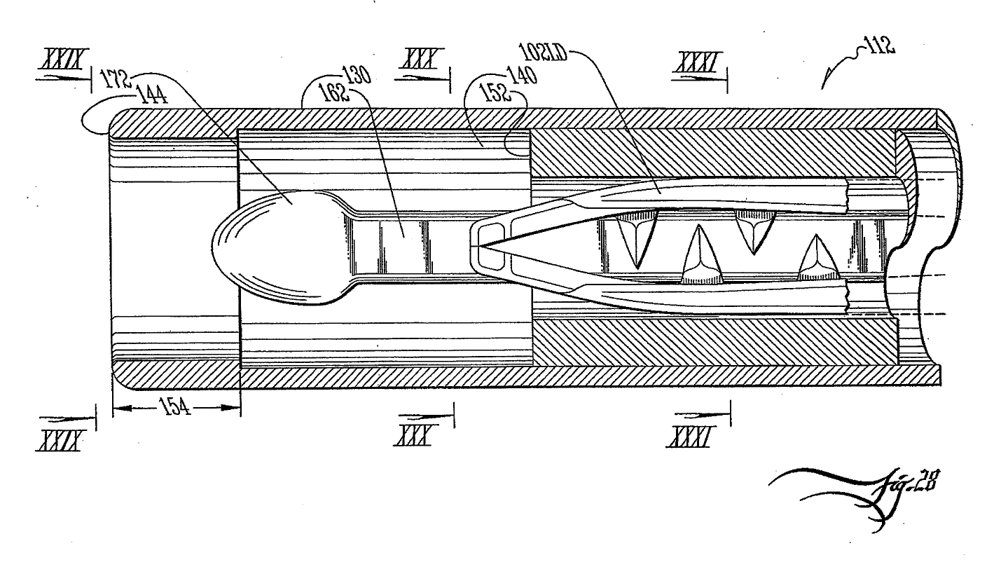

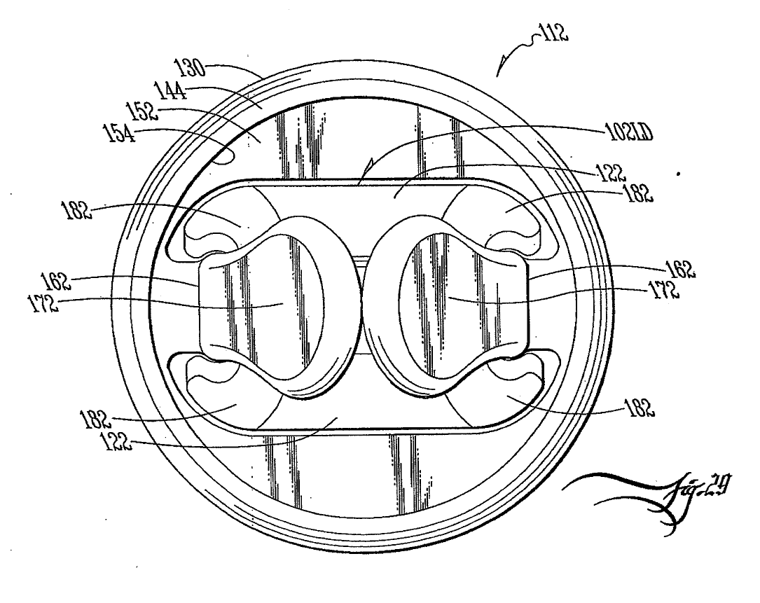

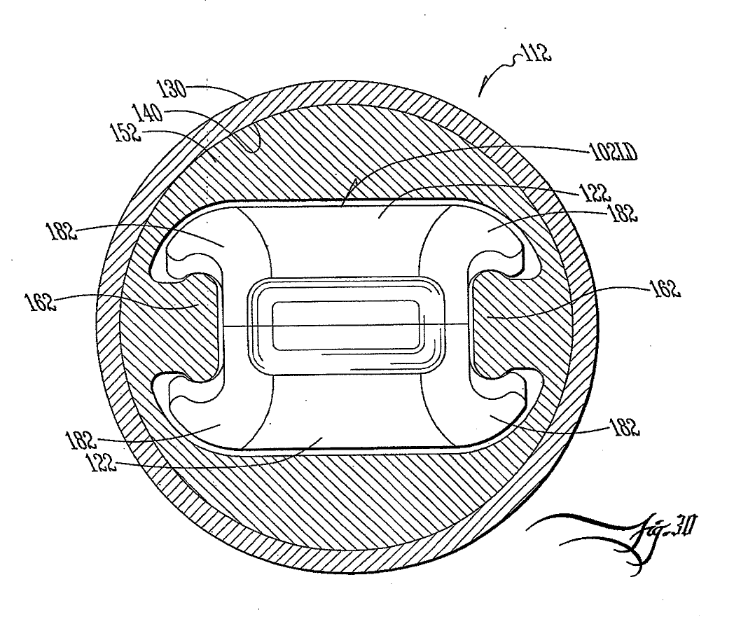

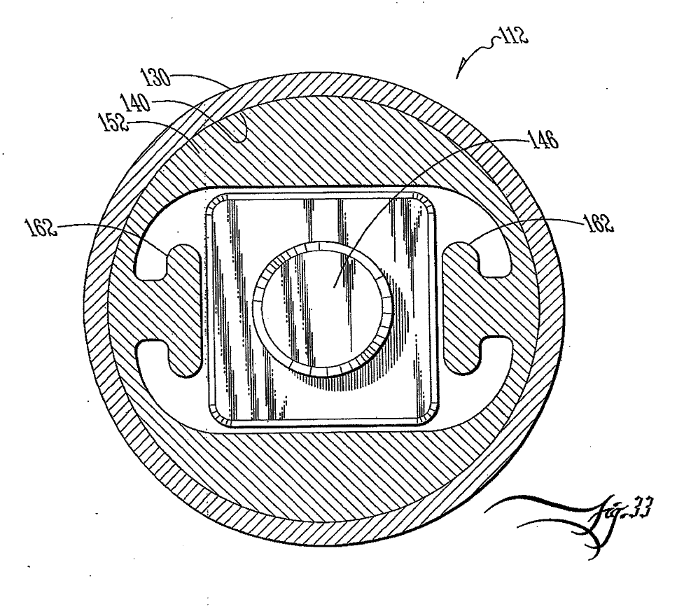

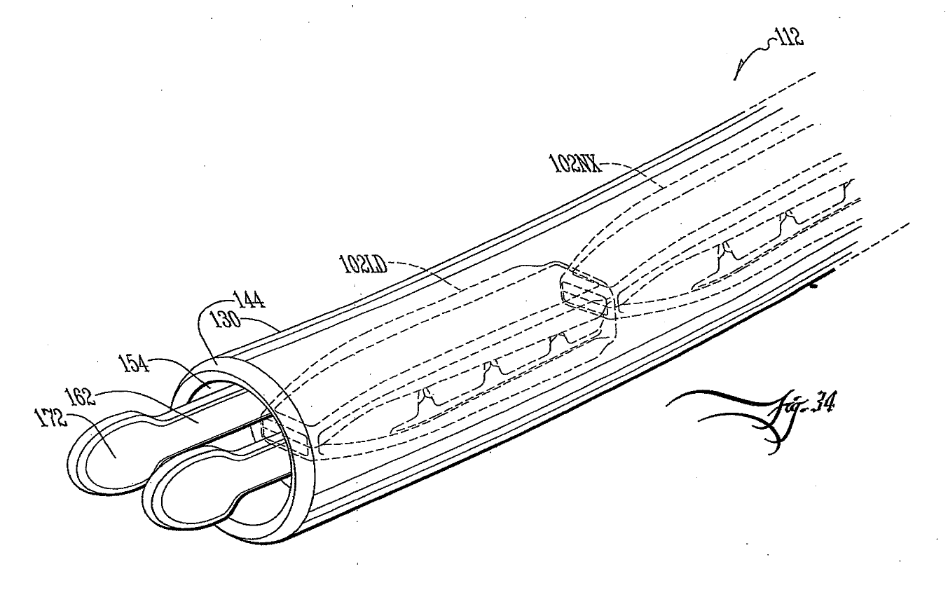

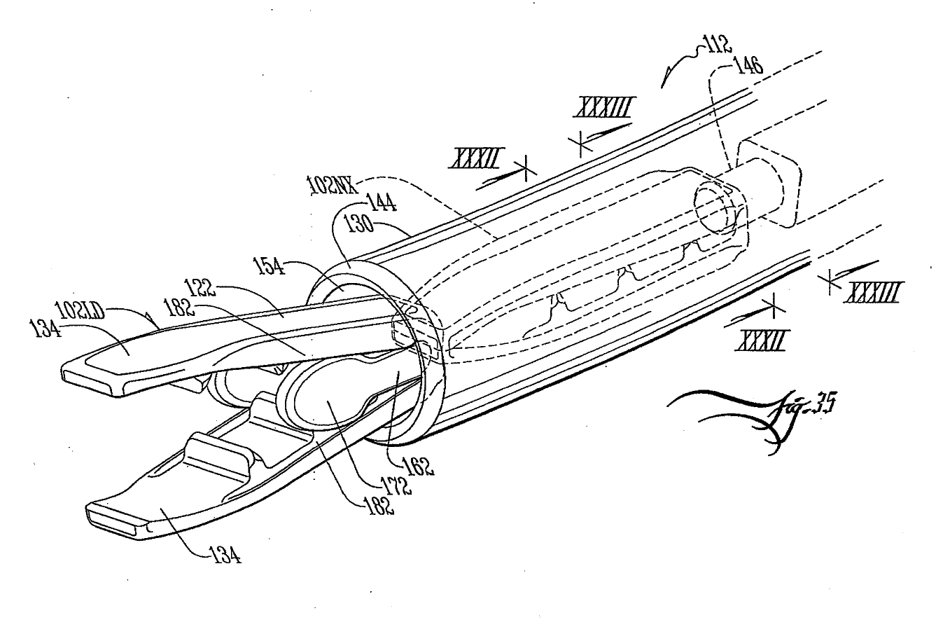

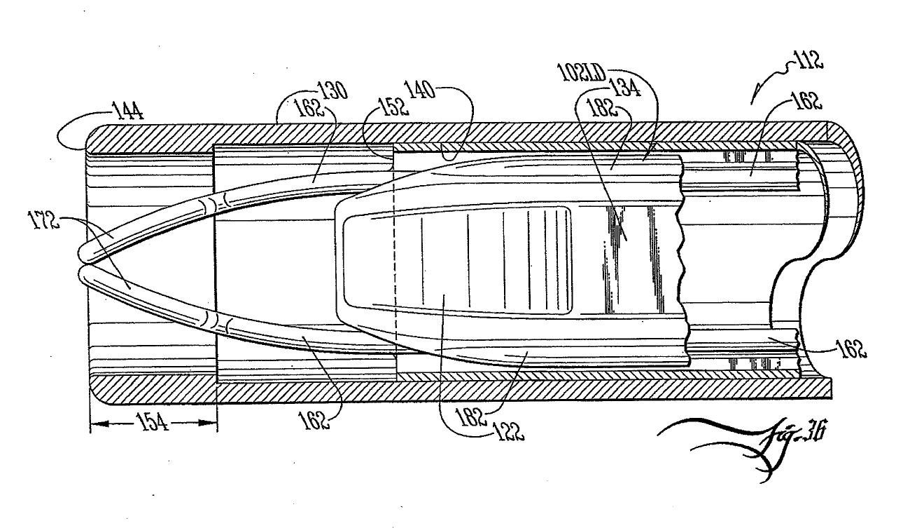

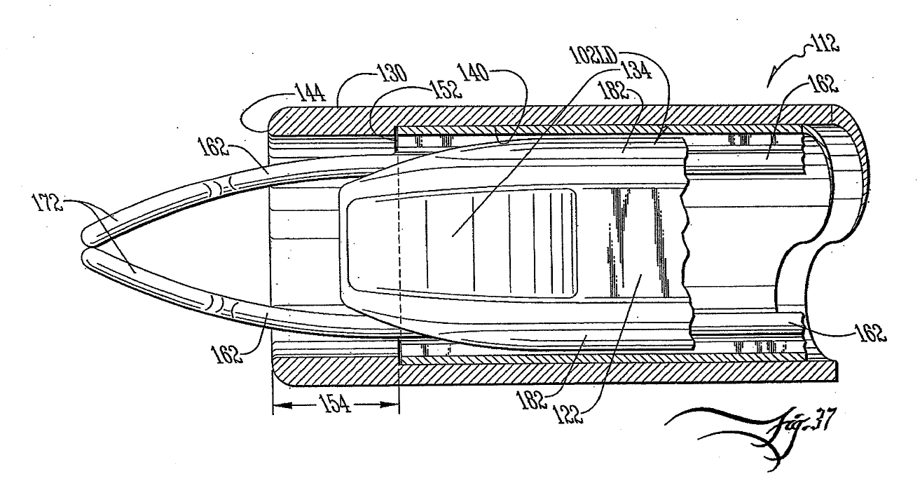

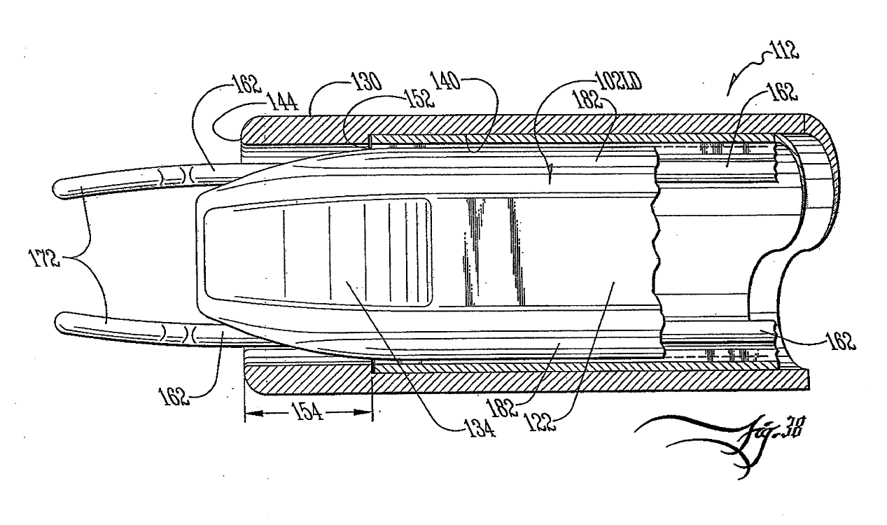

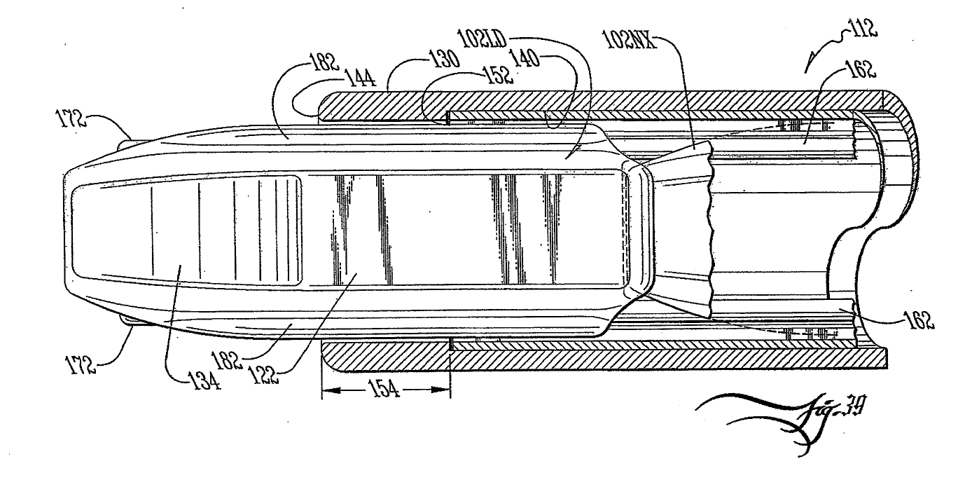

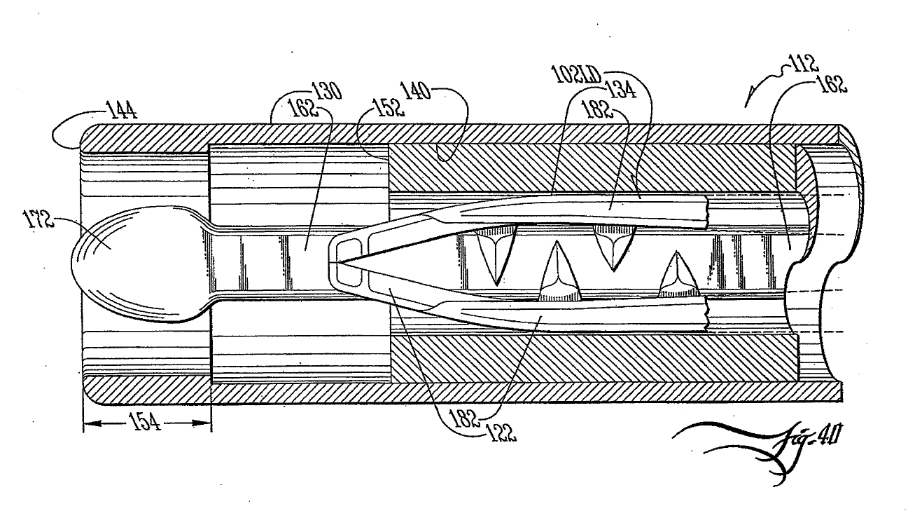

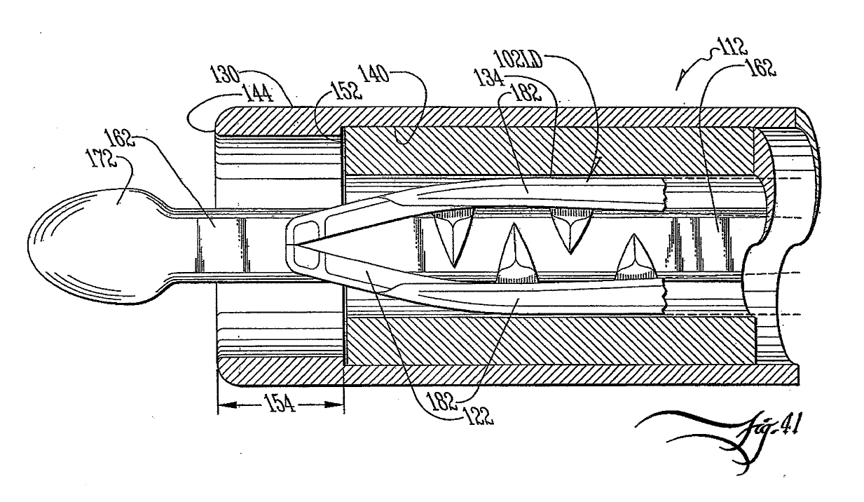

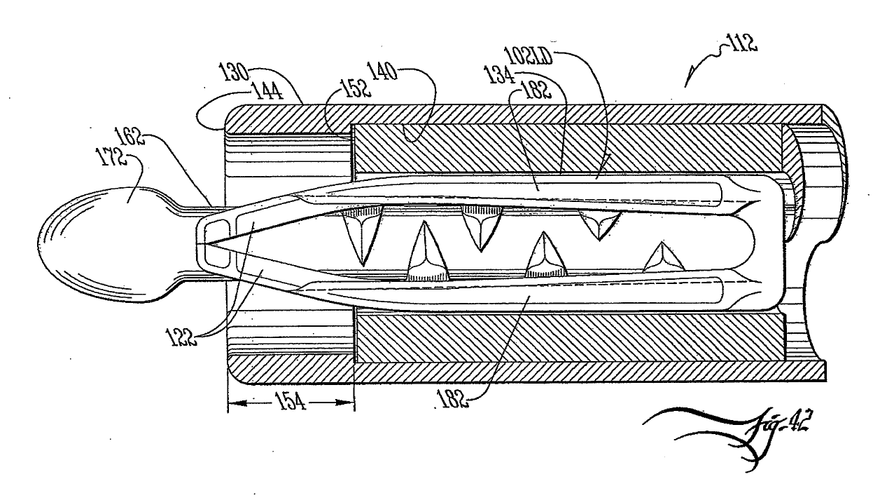

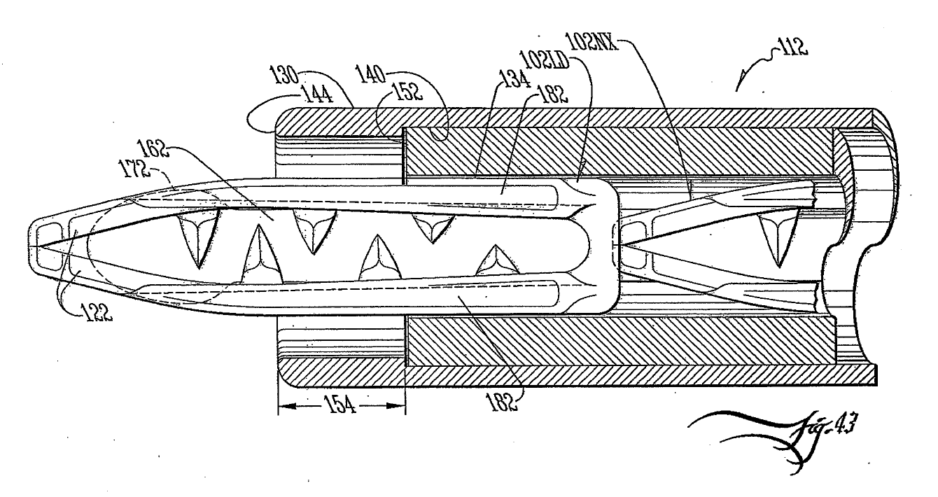

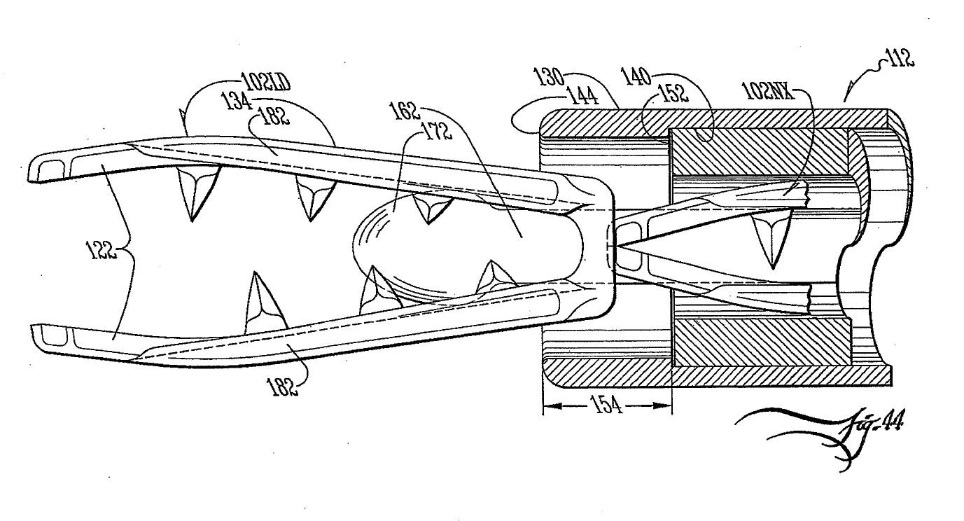

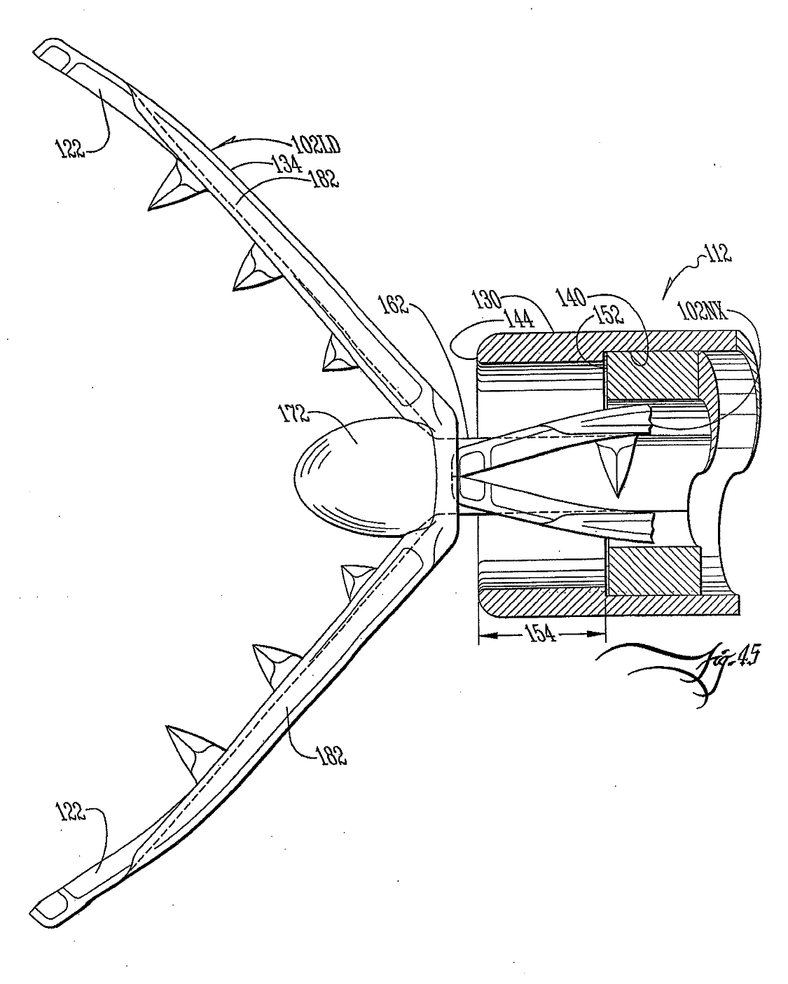

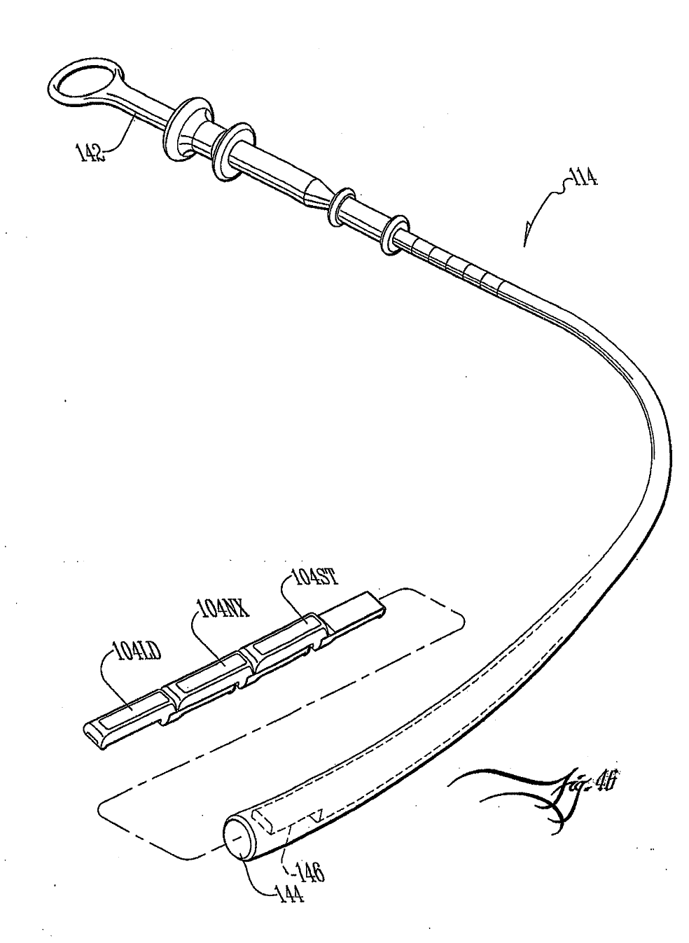

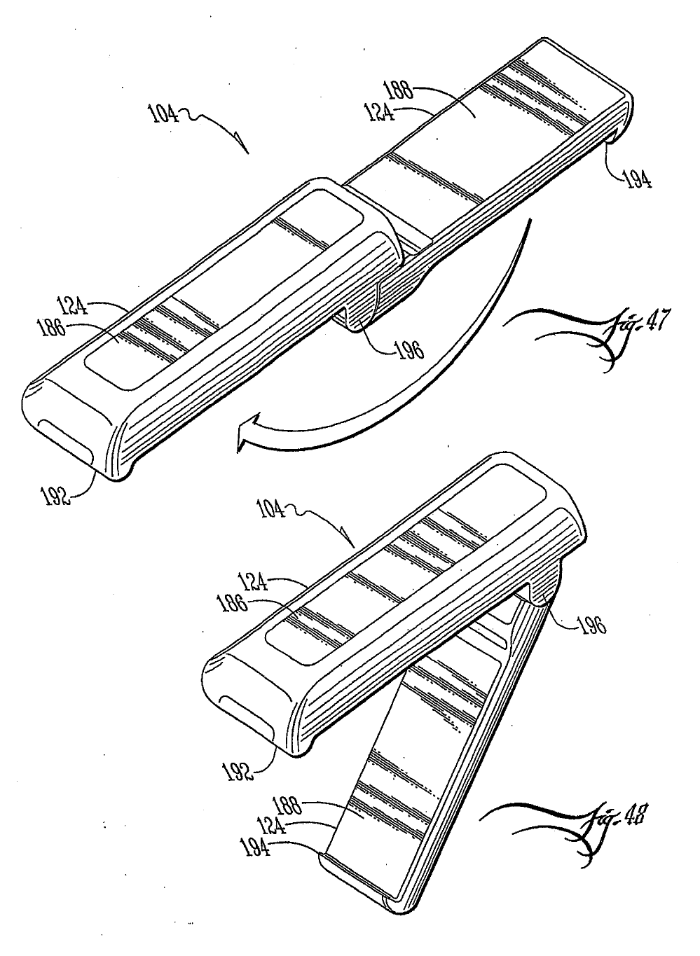

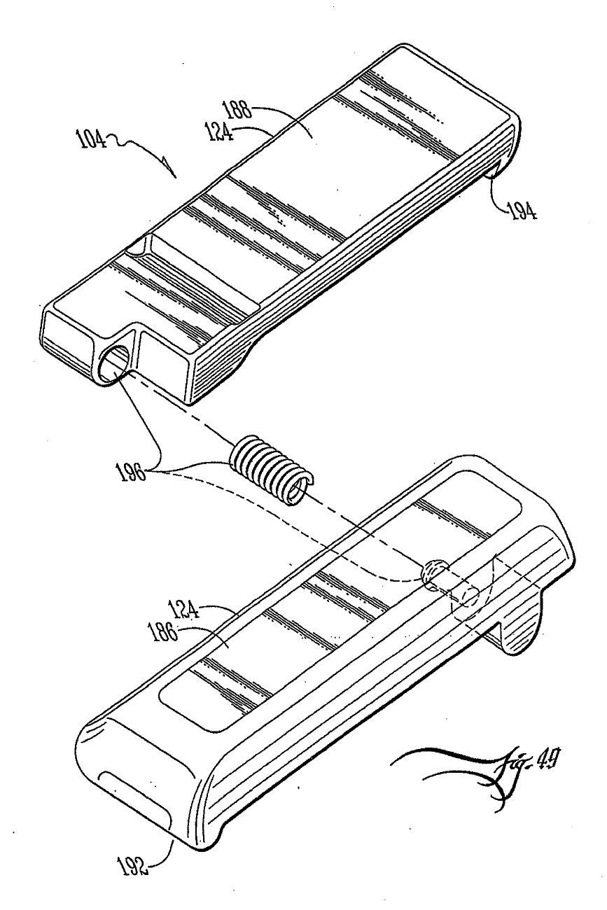

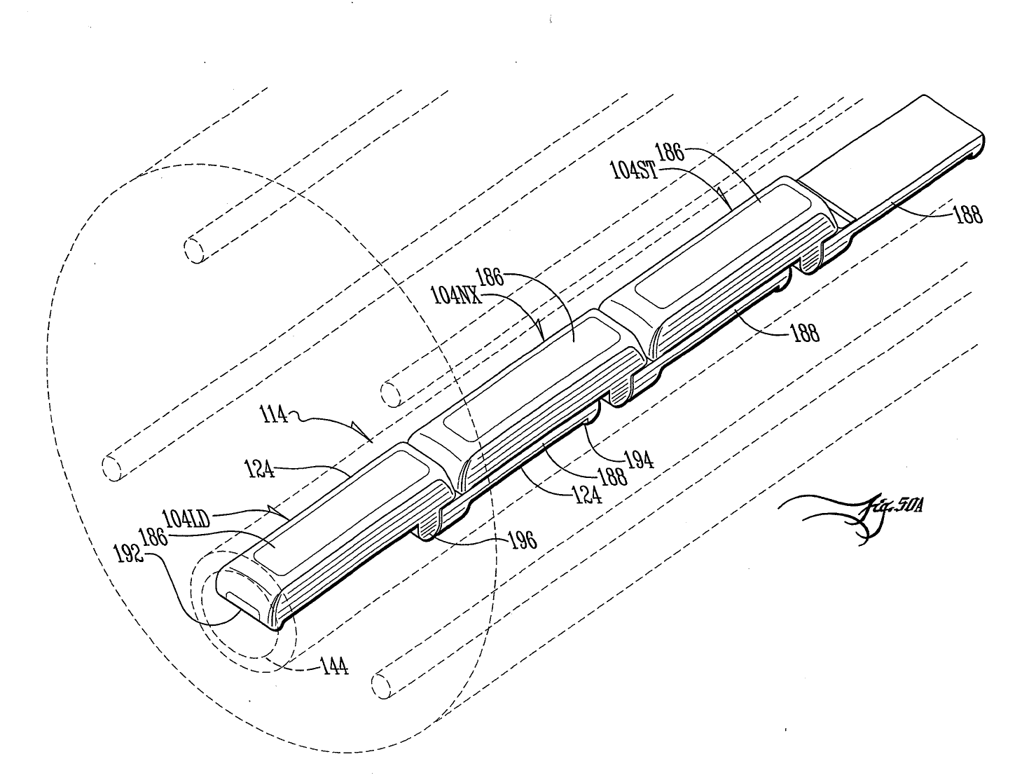

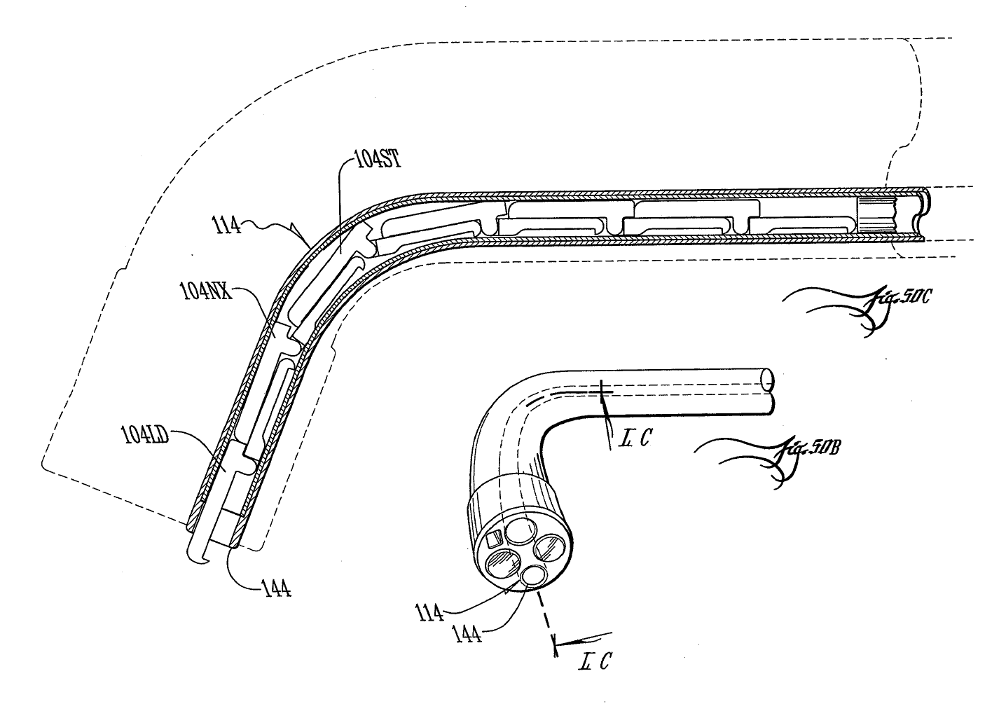

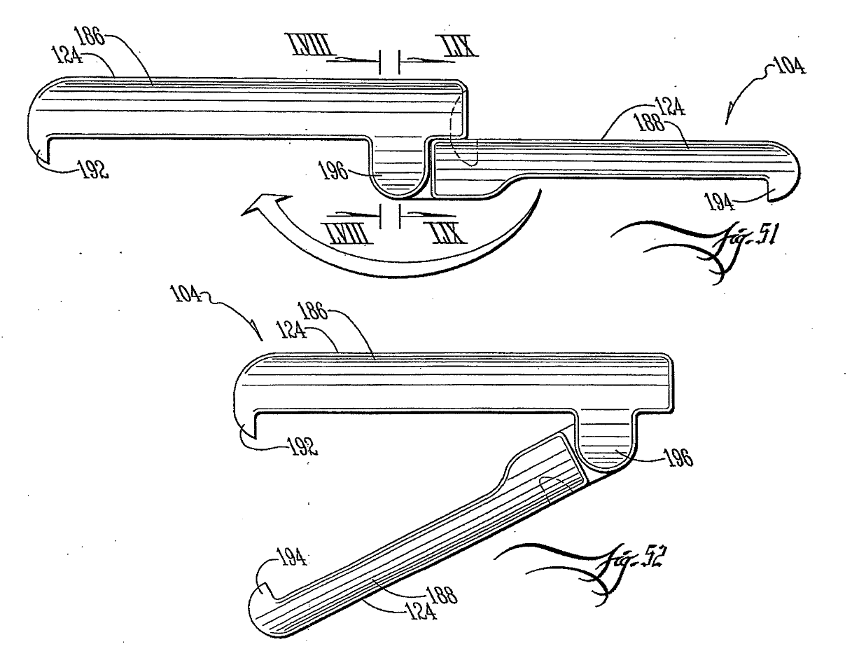

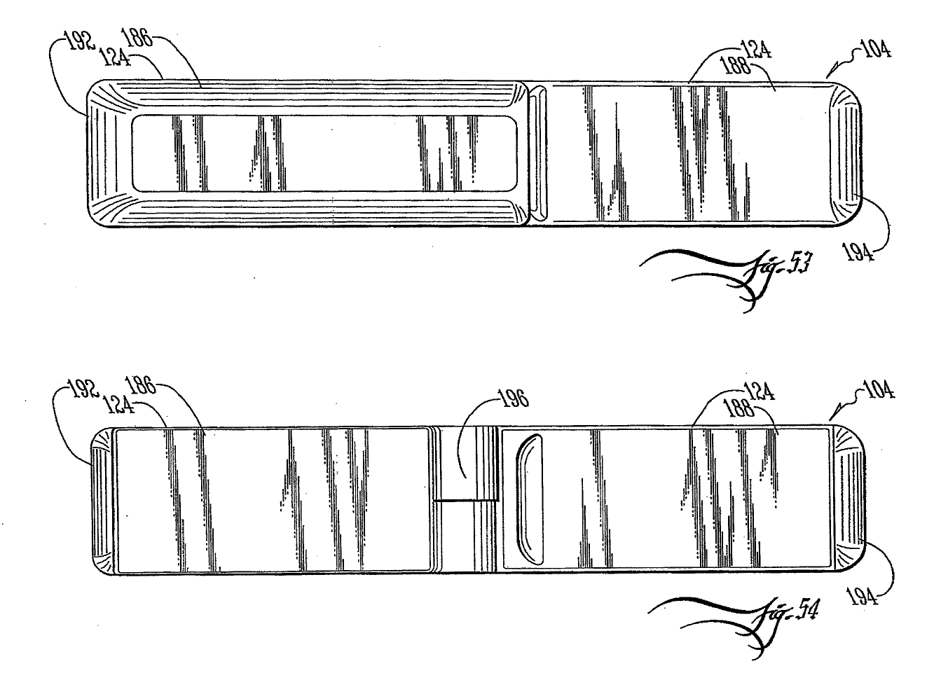

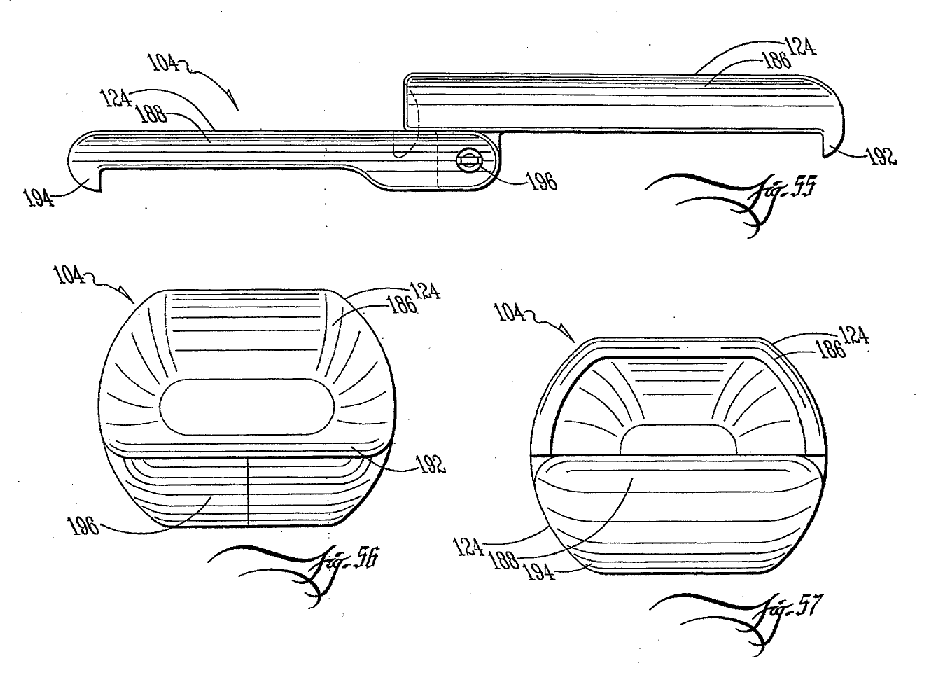

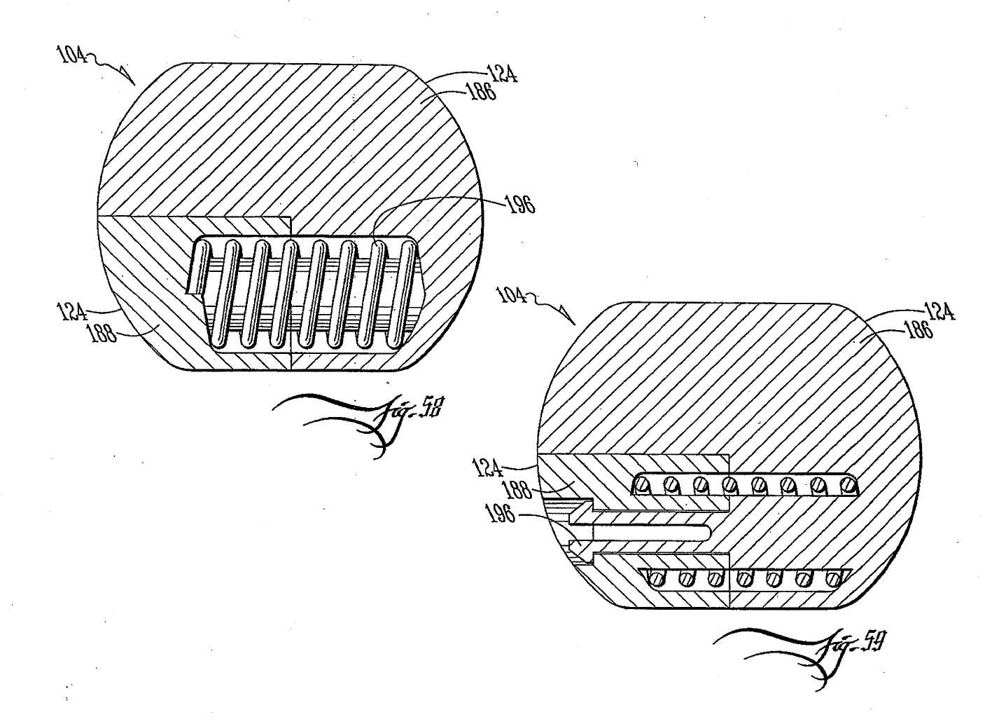

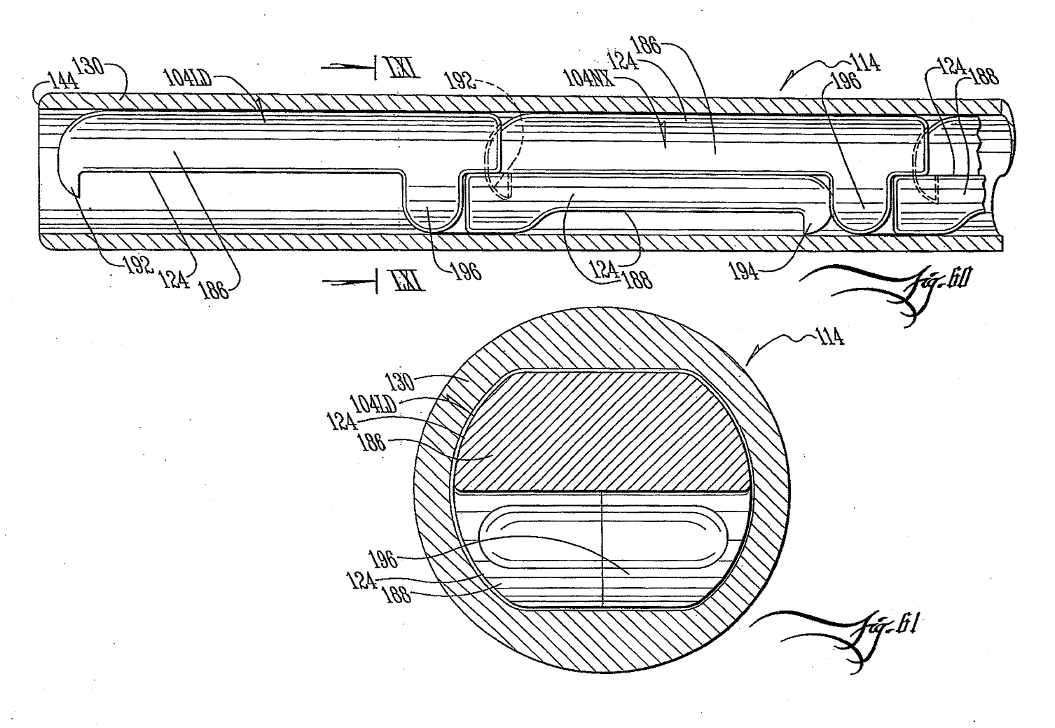

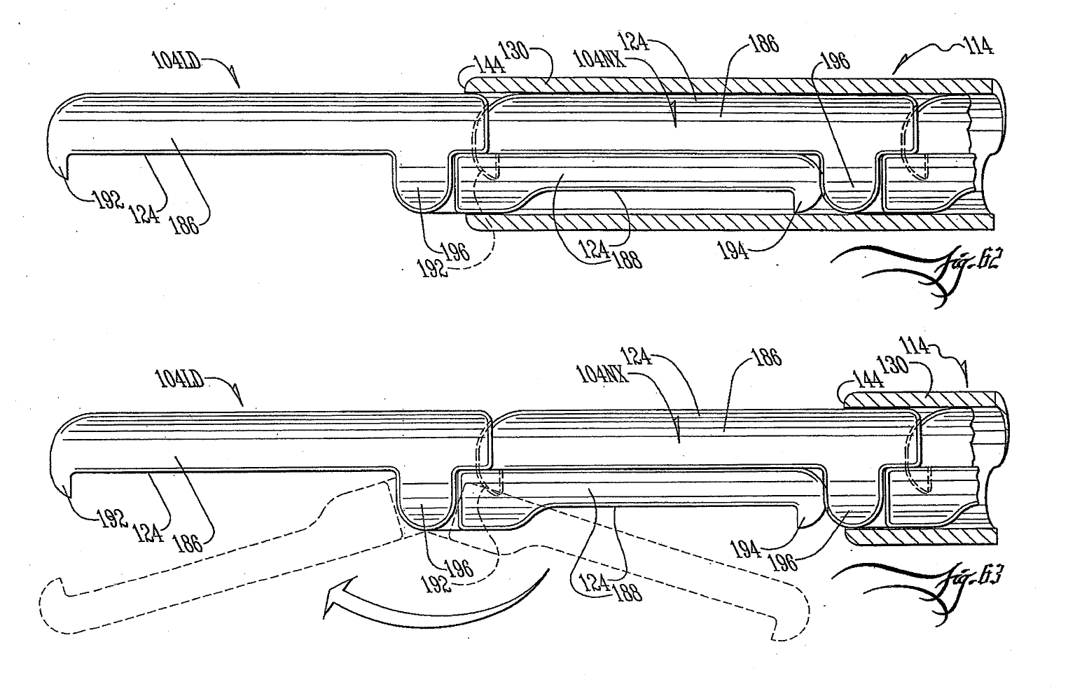

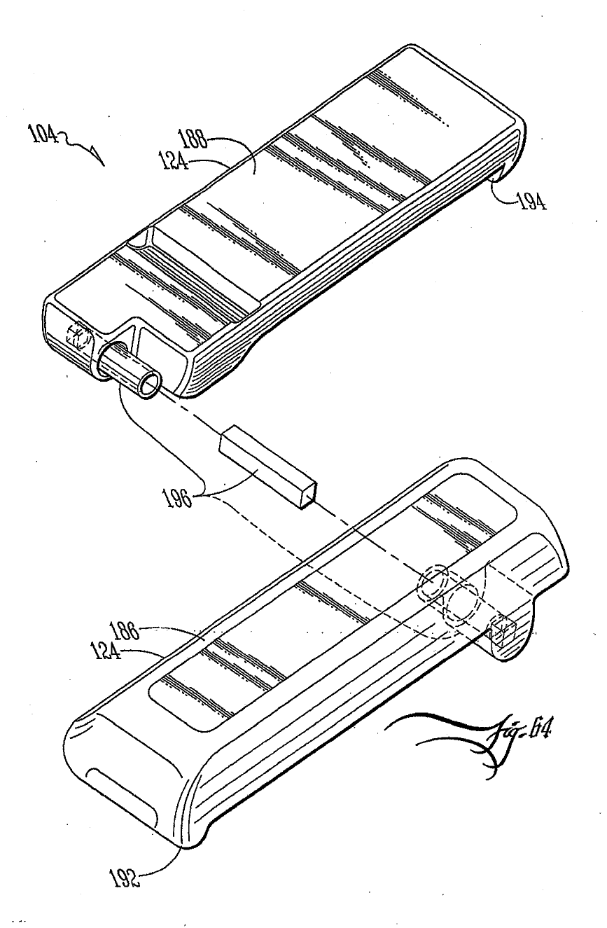

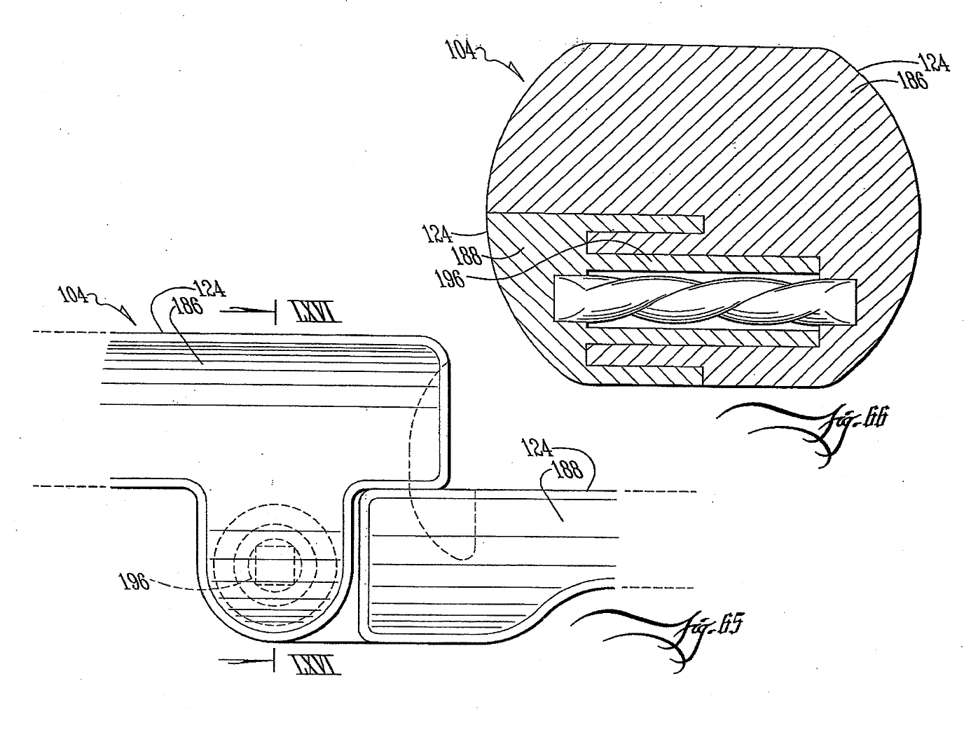

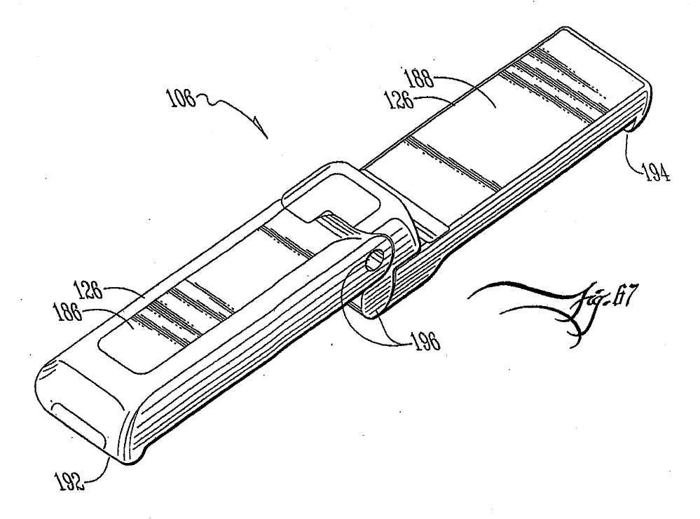

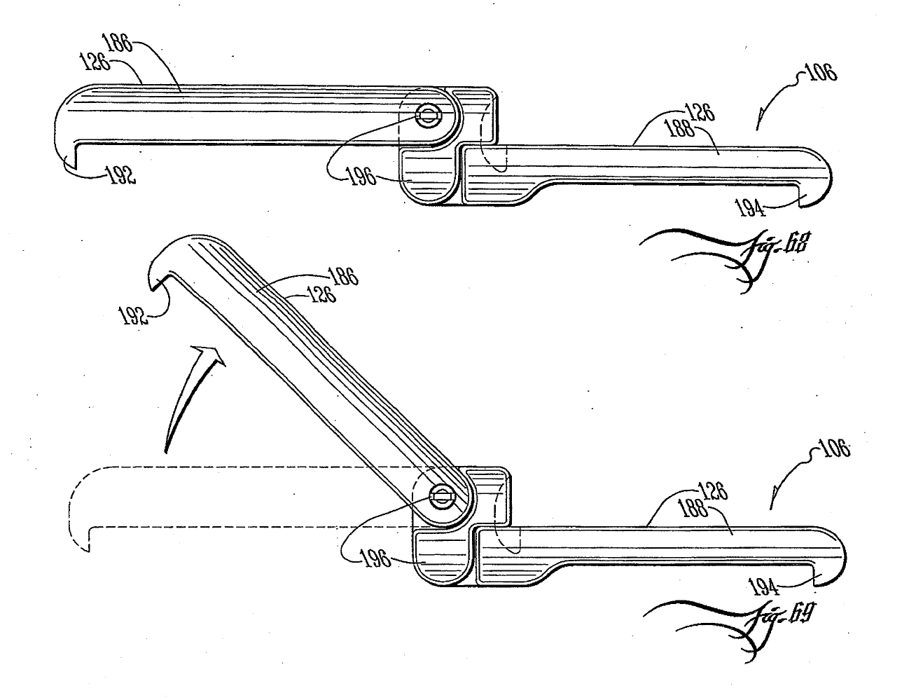

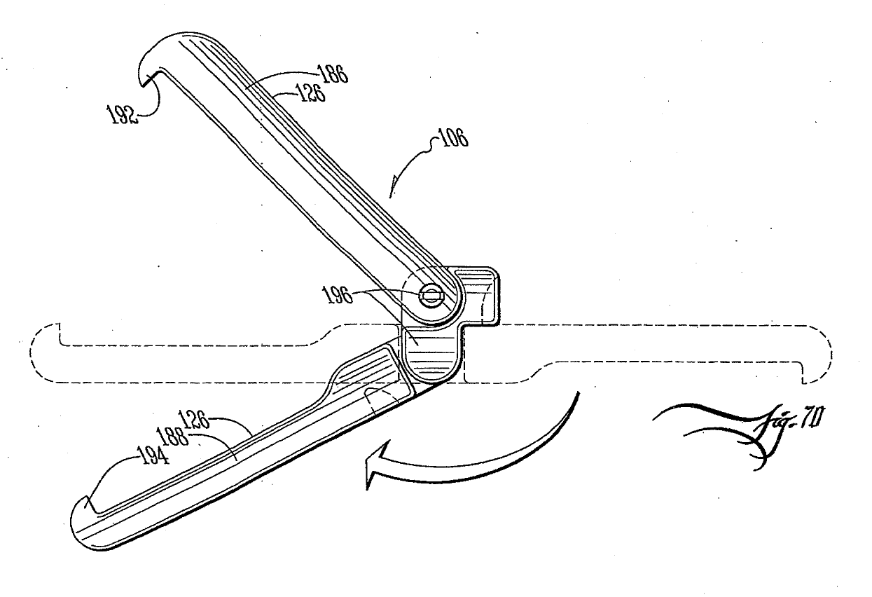

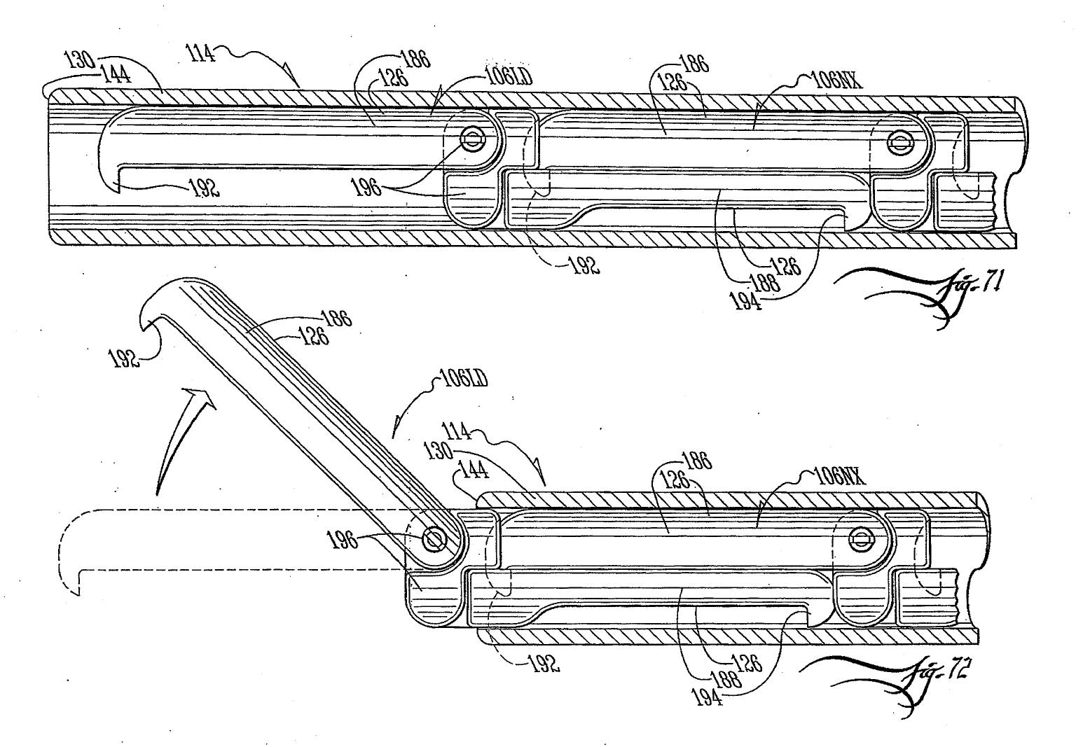

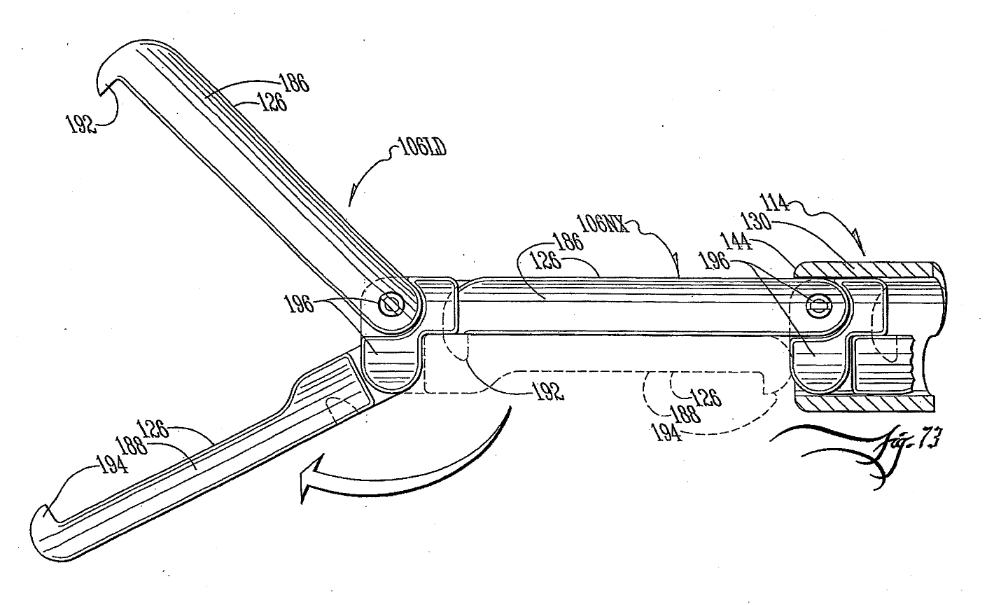

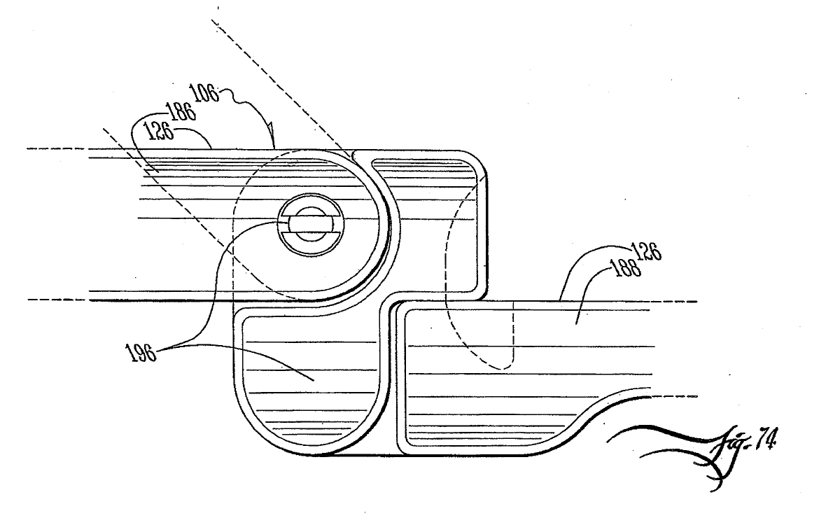

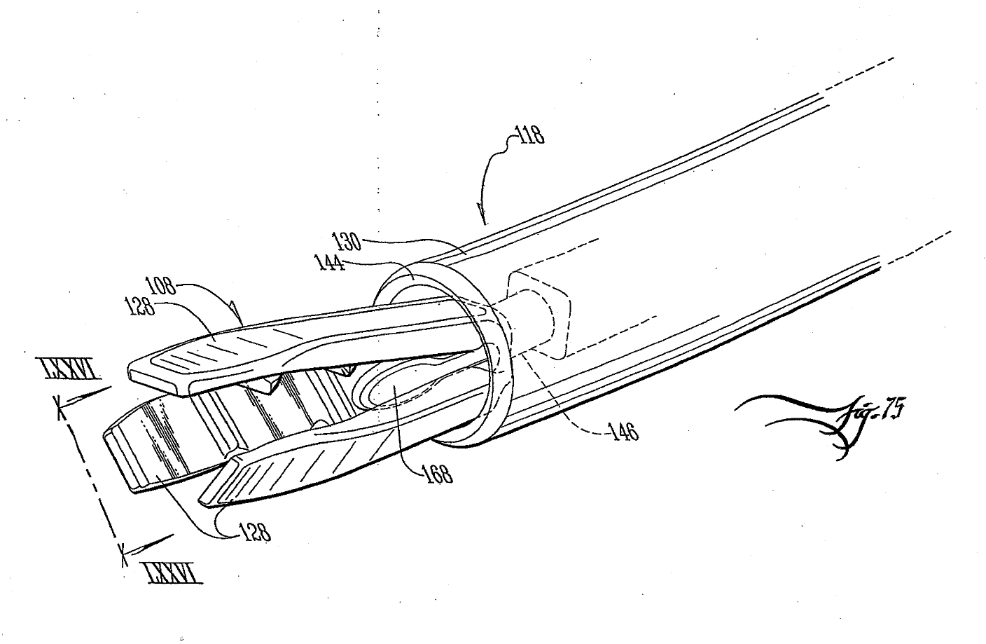

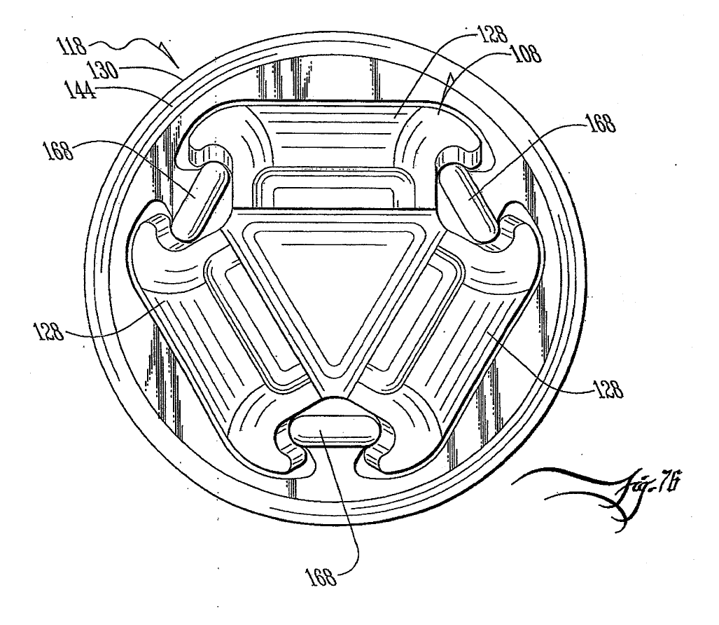

Referring to FIGS. 1A-19 and 20-45 show a first and second embodiment respectively wherein the clips 100 and 102 have a pair of jaws 120 and 122, respectively, which face forwardly when loaded in the dispensing apparatus 110 and 112, respectively. FIGS. 75 and 76 comparably discloses a third embodiment of a clip 108 when loaded in the dispensing apparatus 118. FIGS. 46-74 will be dealt with more particularly below.

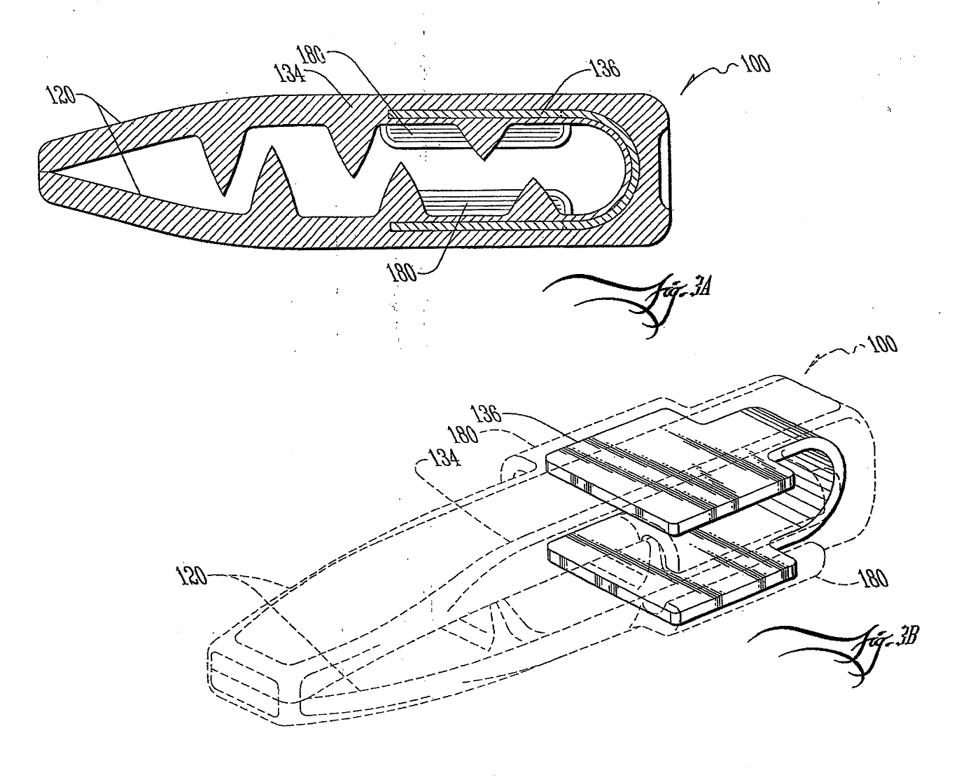

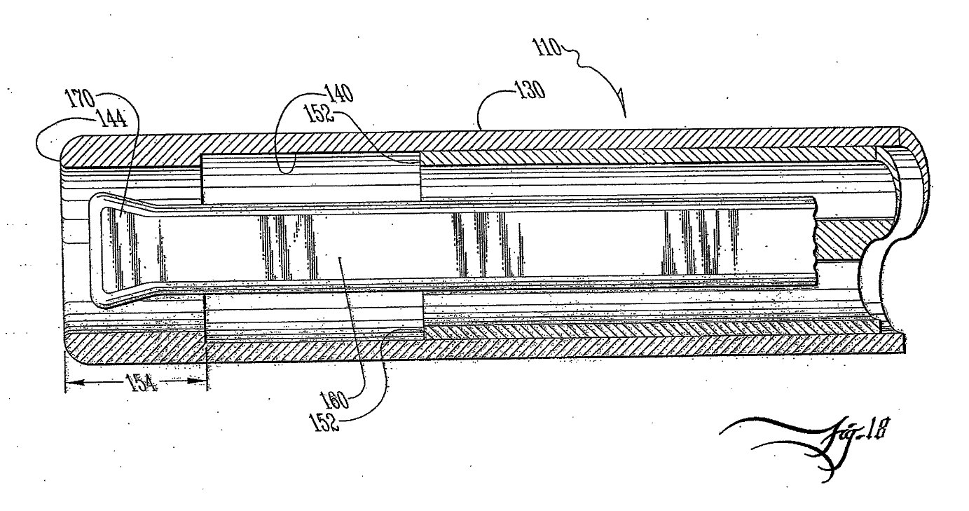

With general reference to FIGS. 1A-45, the clip 100 or 102 may comprise a composite construction 134 and 136 comprising tissue biting jaws permanently biased shut by a clamping spring 136 which has a C-shaped profile from the side (see, eg., FIG. 3B). Alternatively, the clips 110 or 102 may comprise a monolithic construction of a single material 134 (see, eg., FIGS. 21-24).

The endoscopic multi-clip dispensing apparatus 110 or 112 in accordance with the invention preferably comprises a sleeve inside a sleeve construction, with an inner tubular conduit 140 inserted inside an outer tubular conduit 130. The outer tubular conduit 130 serves as the frame of reference for the actuation and movement of the other parts of the multi-clip dispensing apparatus 110 or 112. The outer tubular conduit 130 originates in a manual actuator 142 and terminates in a dispensing end 144.

The inner tubular conduit 140 has a reciprocation action inside the outer tubular conduit 130, reciprocating between extended and retracted extremes. The inner tubular conduit 140 has a sidewall defining an axial lumen. The series of clips 100 or 102 are loaded in the axial lumen of the inner tubular conduit 140. The series of clips are pushed from behind by an elongated flexible plunger rod 146 serving as a pressure-urging follower. The plunger rod 146 can be indexed through the inner tubular conduit 140 in increments corresponding to the axial length of each clip 100 or 102. That way, the plunger rod 146 pushes forward the line of clips 100 or 102 the increment of one clip's length at a time. Thus the plunger rod 146 forces the dispensing of a single clip 100 or 102 at a time.

The plunger rod 146 is indexed continually forward at one increment at a time until the last clip 100ST or 102ST is dispensed. Then the plunger rod 146 may be retracted all the way back to an original starting point, such as for (needless to say) the reloading of a fresh line of clips 100 or 102. This can be likened to and without limitation as the way a caulk gun operates on a tube of caulk. The caulk gun plunger rod is ratcheted continually forward in the tube of caulk, forcing the dispensing of caulk out the dispensing end. Once the caulk gun plunger rod reaches the end, and the tube of caulk is depleted, the caulk gun plunger rod is released to be retracted all the way back to the starting point. The depleted tube of caulk is discarded, and a fresh tube of caulk is loaded.

As mentioned, the inner tubular conduit 140 has a reciprocation cycle inside the outer tubular conduit 130. The inner tubular conduit 140 normally rests in its retracted extreme. During an actuation cycle, the inner tubular conduit 140 extends through an extension stroke to an extended extreme. It momentarily holds stationary in the extended extreme as the lead clip 100LD or 102LD is ultimately ejected, after which, the inner tubular conduit 140 returns to rest in its retracted extreme.

The inner tubular conduit 140 has a terminal end 152 which is axially spaced away and inside of the dispensing end 144 of the outer tubular conduit 130. Hence, resting in the retracted extreme, the terminal end 152 of the inner tubular conduit 140 is gapped away from the dispensing end 144 and inside the core of the outer tubular conduit 130 by a given distance. During the actuation cycle, the terminal end 152 of the inner tubular conduit 140 travels about half that given distance to come closer to the dispensing end 144 of the outer tubular conduit 130. But, the terminal end 152 of the inner tubular conduit 140, when reaching the extended extreme, does not travel so far as to actually protrude out of the dispensing end 144.

Hence the foregoing defines an “ejection station” 154 inside the dispensing end 144 of the outer tubular conduit 130. That is, the axial length in the core of the outer tubular conduit 130 between its dispensing end 144, and, the terminal end 152 of the inner tubular conduit 140 in the extended extreme, defines the “ejection station” 154.

It is in the ejection station 154 that the lead clip 100LD or 102LD is ultimately spread open, ejected, and allowed to form a “bite” of clamping pressure on some target tissue:—which target tissue is targeted by the operator of said endoscopic multi-clip dispensing apparatus 110 or 112 in accordance with the invention (eg., a surgeon).

While the inner tubular conduit 140—speaking strictly of it alone—does not extend out the dispensing end 144 of the outer tubular conduit 130, the inner tubular conduit 140 does have a pair of arms 160 or 162:—which do. That is, the inner tubular conduit 140 has protruding from its terminal end 152 a pair of arms 160 or 162 (at least a pair of arms). That is, if the clips 100 or 102 only have a pair of jaws 120 or 122 (eg., see, FIGS. 1A-45) then the inner tubular conduit 140 will have just a pair of arms 160 or 162. However, if the clips 108 have three jaws 128 (eg., see, FIGS. 75 and 76), then the inner tubular conduit 140 will have three arms 168. And so on, the number of arms roughly corresponding to the number of jaws.

The arms 160 or 162 project from base ends anchored in the inner tubular conduit 140 proximate the terminal end 152 thereof, to free ends 170 or 172. When the inner tubular conduit 140 is resting in its retracted position, the free ends 170 or 172 of the arms 160 or 162 lie just inside the dispensing end 144 of the outer tubular conduit 130. When the inner tubular conduit 140 is extended to its extended extreme, the free ends 170 or 172 of the arms 160 or 162 do indeed project outside of the dispensing end 144 of the outer tubular conduit 130.

The free ends 170 or 172 of the arms 160 or 162 are formed in wedge-shaped flared ends 170 or 172, which also are flexed in toward each other, each at the end of a respective arm 170 or 172. Preferably the arms 160 or 162 are produced out of not only a resilient material but a durable material too, one which resists erosion after the scraping passage thereover of numerous clips 100 or 102. It is preferred without limitation that the arms 160 or 162 are produced of spring steel.

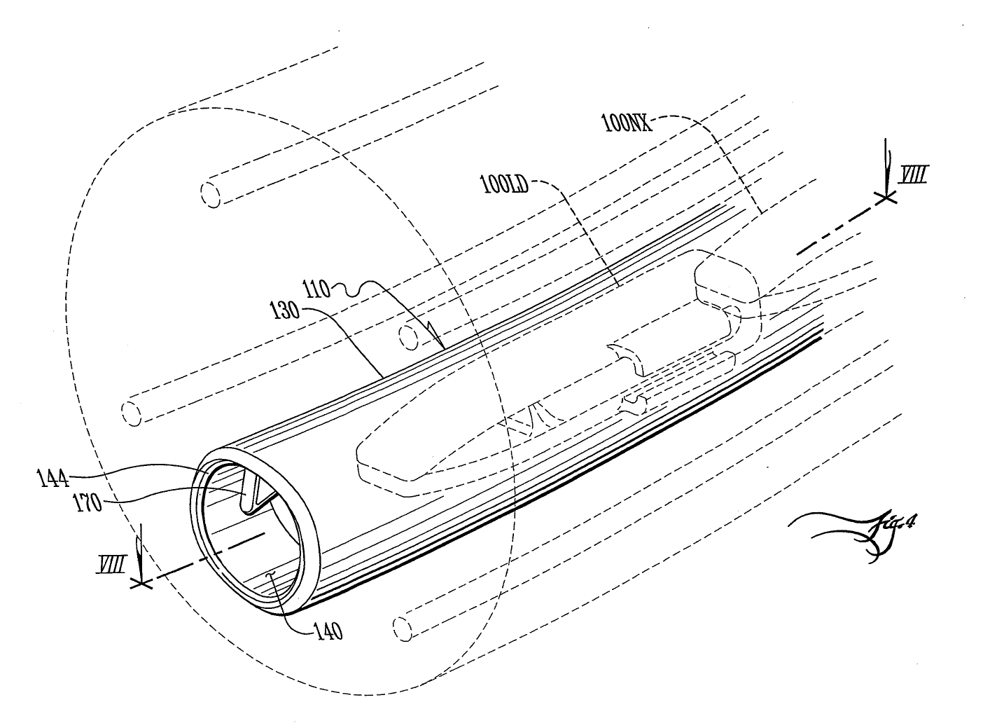

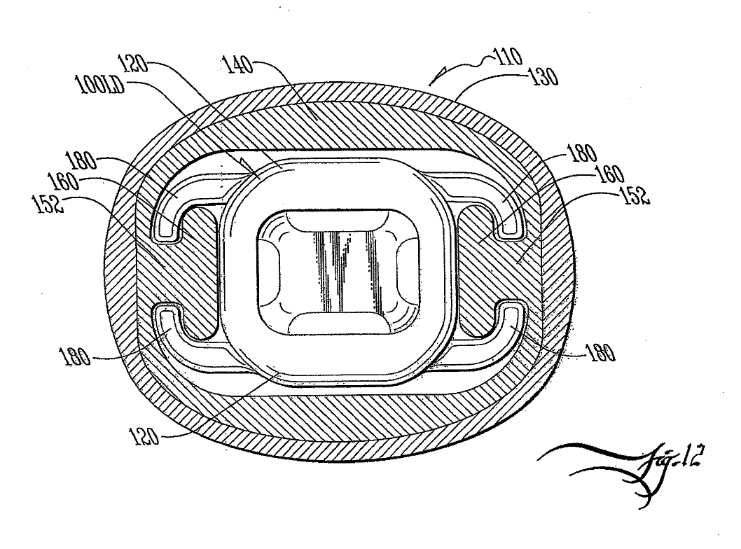

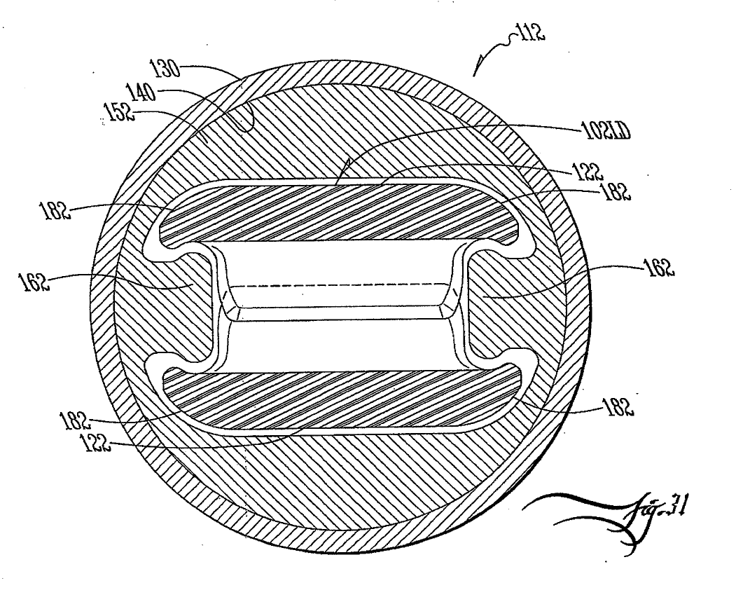

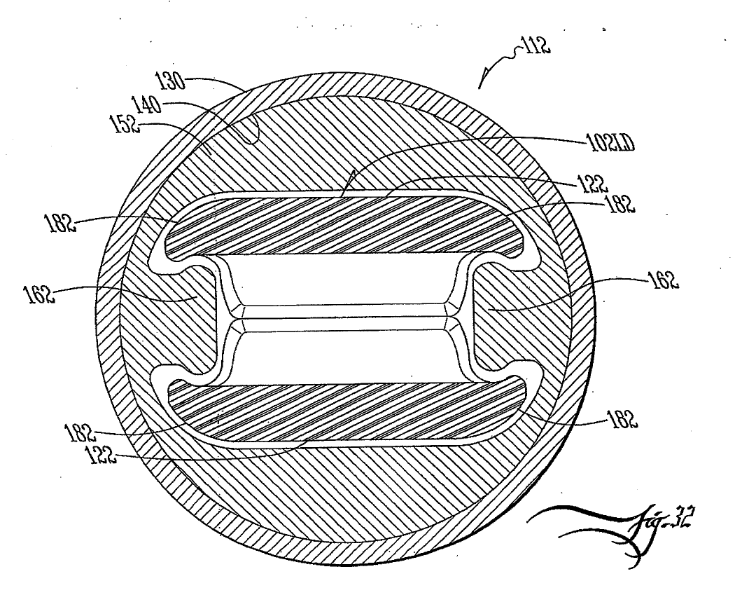

The drawings figures provide several sequences of views showing how one clip 100 or 102 is ultimately dispensed. See, eg., FIGS. 4-7, and/or FIGS. 13-17, and/or FIGS. 34-35, and/or FIGS. 36-39, and/or FIGS. 40-45, and so on.

Therefore, any of the foregoing sequences of views show a progression comprising the lead clip 100LD or 102LD of a series of clips 100 or 102, respectively, in accordance with the invention being dispensed out the dispensing end 144 of the endoscopic multi-clip dispensing apparatus 110 or 112, respectively, in accordance with the invention.

The dispensing apparatus 110 or 112 is shown in section in the various sequences identified above, and from various perspectives, or from vertical and horizontal cutting planes through the longitudinal axis thereof. The series of clips 100 or 102 are typically shown in a head to tail line up (ie., wherein the head end is lead by the jaws 102 or 122 and the tail end is where the spring 136 is disposed), which each sequence beginning with the lead clip 100LD or 102LD in position for dispensing.

The sequences show the progression of changes the lead clip experiences through the process of being dispensed. The lead clip 100LD or 102LD is being pushed from behind by a considerable thrusting force on the line of clips 100 or 102, respectively, in the dispenser's inner tubular conduit 140. As do all of the clips 100 or 102, the lead clip 100LD or 102LD has upper and lower jaws 120 or 122, respectively, giving the clip 100LD or 102LD an overall C-shape. Both the upper and lower jaws 120 or 122, respectively, have turned-in flanges or turned-in channels defining eaves 180 or 182, respectively, or tracks that ride along the edges up the wedge-shaped flared ends 170 or 172 of the arms 160 or 162, respectively. With the eaves or tracks 180 or 182 of the C-shaped clip 100LD or 102LD, respectively, being spread open by scraping transit over the wedge-shaped flared ends 170 or 172 of the arms 160 or 162, respectively, the tissue biting jaws 120 or 122 of the lead clip 100LD or 102LD open, and, open wide.

Moreover, the whereby the arms of the tubular conduit and flanges of the lead clip cooperate to prevent relative spinning.

In order for the jaws 120 or 122 to open that wide, the wedge-shaped flared ends 170 or 172 of the arms 160 or 162 have to reciprocate through a cycle of extension and retraction strokes. The flared ends 170 or 172 extend outside of the dispensing end 144 of the outer tubular conduit 130 in order to give the tail end of the clip 100LD or 102LD sufficient clearance to flex open wide. Otherwise, if confined in the ejection station 154, the lead clip 100LD or 102LD would not be able to open as wide. As the eaves or tracks 180 or 182 of the lead clip 100LD or 102LD ultimately pass by the flared ends 170 or 172 of the extended arms 160 or 162, the jaws 120 or 122 of the lead clip 100LD or 102LD are free to snap shut. At the same time, the lead clip 100LD or 102LD is released from any further contact with the dispenser 110 or 112 or line of trailing clips 100 or 102. The lead clip 100LD or 102LD is free to bite firmly on any tissue which the operator has aimed the lead clip 100LD or 102LD at (eg., an example operator is a surgeon). In the last stage of the ejection of the lead clip 100LD or 102LD, the line of trailing clips 100 or 102 from behind move forward until what becomes the succeeding new lead clip is trapped in a ready-to-go, next-to-be ejected position by the retracting arms 160 or 162 and inward flexing flared ends 170 or 172 of inner tubular conduit 140. That is, the wedge-shaped flared-ends 170 or 172 retract by the retraction stroke of the inner tubular conduit 140 and form somewhat of a clapped together embrace with each other by the resiliency in the arms 160 or 162. This clapping embrace of the flared ends 170 or 172 can be likened to, and without limitation, a single clap of a person's hand.

To turn now to FIGS. 46-74, these views show several embodiments of clips 104 and 106, and, dispensing apparatus 114 arranged wherein the jaws 124 or 126, respectively, open to about 180° apart when loaded in the tubular conduit 140 of the dispensing apparatus 114. In such an arrangement, one jaw 124 or 126 is the lead jaw 186 and the other jaw 124 or 126 is the trailing jaw 188. The leading jaw 186 has a leading hook-end 192 and the trailing jaw 188 has a trailing hook-end 194. During the progression of dispensing the leading clip 104LD or 106LD out of the dispenser end 144, the leading clip 104LD or 106LD′s leading end 192 extends out first. Preferably the leading hook-end 192 of the leading jaw 186 is latched onto some target tissue (not shown) so the that clip 104LD or 106LD does not wastefully close on itself without clamping any tissue. Then when the trailing jaw 188 clears the dispensing end 144 of the dispensing apparatus 114, the trailing jaw 188 snaps shut toward the leading jaw 186, and thereby forms the intended clamping action.

The dispensing apparatus 114 only needs a single tubular conduit 130, and not a sleeve-inside-a sleeve configuration as before. However, the dispensing apparatus 114 still needs a plunger rod (not shown) incrementally forcing each clip 104 or 106 in series out of the dispensing end 144, one clip at a time. Preferably any give clip 104 or 106 ahead of another in the line of clips 104 or 106, respectively, is pushed forward by the leading end 192 of the trailing clip 104 or 106 bearing against the hinge 196 of the leading clip 104 or 106, respectively.

It is further preferred if the core of the tubular conduit 130 had a metering device (not shown) such as and without limitation a miniature turn-stile or the like proximate the dispensing end 144. The metering device would preferably meter the dispensing of the clips 104 or 106 to one clip at a time. That is, as soon as the current-lead clip 100LD or 106LD is ejected, the metering device holds back or offers resistance to the next clip 104NX or 106, NX, respectively. However, the means holding back the next clip 104NX or 106NX or offering the resistance can be overcome after a moment's wait to thereby allow the operator (eg., surgeon) to eject the next clip.

The invention having been disclosed in connection with the foregoing variations and examples, additional variations will now be apparent to persons skilled in the art. The invention is not intended to be limited to the variations specifically mentioned, and accordingly reference should be made to the appended claims rather than the foregoing discussion of preferred examples, to assess the scope of the invention in which exclusive rights are claimed.

I claim:

1. A combination endoscope, endoscopic multi-clip dispensing apparatus and a plurality of clips therefor, comprising:
an elongated flexible endoscope extending between an operator end and a probe end;
said elongated endoscope comprising at least an internal optics channel with a lens proximate the probe end, a source of illumination proximate the probe end, control wires that control the flexure of the endoscope, and an internal working channel having a forward opening in the probe end and a rearward opening proximate the operator end;
an elongated flexible inner tubular conduit sleeved inside an elongated flexible outer tubular conduit, wherein the inner tubular conduit reciprocates between extension and retraction strokes;
a plurality of clips, each having a C-shape characterized by a pair of elongated jaws mated together at a hinge end, wherein the jaws have tip ends forming a head end for the clip and hinge end forms a tail end for the clip when said plurality of clips are loaded into a core of the inner tubular conduit in head to tail fashion;
an elongated flexible plunger rod for pushing the line of clips through the core of the inner tubular conduit at an incrementally indexed or metered rate of one clip at a time; and
a pair of wedge-shaped flared structures proximate the dispensing end of one of the outer tubular conduit or inner tubular conduit for prying open the jaws of a lead clip during a progression of the lead clip being dispensed whereby, after the lead clip passes clear of the flared structures, the jaws thereof are free to snap shut and ‘bite’ into a target anatomical structure;
wherein the plurality of clips when loaded into the core of the inner tubular conduit in head to tail fashion allow relative articulation between each clip whereby, with all of the elongated inner tubular conduit and the elongated outer tubular conduit as well as the elongated plunger rod being flexible, said endoscopic multi-clip dispensing apparatus may being threaded through the working channel of the endoscope and operated thereafter to dispense clips.

2. A combination of an endoscopic clip dispensing apparatus and a clip therefor, comprising:
an elongated tubular conduit defining an axial lumen, terminating in a terminal end;
a pair of opposed slender arms affixed relative the tubular conduit and which project past the terminal end and ultimately terminate in wedge-shaped flared ends that are biased toward each other such that they are at least partly a block across the lumen of the elongated tubular conduit;
each arm having a pair of angularly-spaced axially-extending edges which are parallel to one another except for the wedge-shaped flared end, wherein said edges for said arm flare apart;
an elongated clip having an elongated C-shape characterized by a pair of elongated jaws mated together at a hinge end, wherein the jaws have tip ends forming a head end for the clip and the hinge end forms a tail end for the clip, said clip being loaded into the lumen of the tubular conduit with the head end behind the wedge-shaped flared ends of the tubular conduit and the tail end behind the head end;
said clip having one and another pair of turned-in axially-elongated spaced flanges, one pair flanking one jaw and the other pair flanking the other jaw, each turned-in axially-elongated flange extending between a head end and a tail end, which tail end of the flange is spaced ahead of the tail of the clip; and
an elongated plunger sleeved inside the lumen of the tubular conduit for pushing the clip up to and past the wedge-shaped flared ends which are affixed relative thereto;
wherein said turned-in axially-elongated flanges are configured to clasp and ride on the edges of the arms where parallel and then engage the edges of the opposed wedge-shaped flared ends to wedge open the jaws of the clip until the tail end of the turned-in axially-elongated flanges pass the opposed wedge-shaped flared ends, after which the jaws of the clip are free to snap shut and ‘bite’ into a target anatomical structure.

3. The combination of claim 2, wherein:
said hinge of the clip further comprises a spring of spring steel formed in a C-shape embedded therein.

4. The combination of claim 2, further comprising:
a plurality of said clips loaded into the lumen of the tubular conduit in head to tail fashion to form a line from a lead clip just behind the wedge-shaped flared ends to a last clip against which the elongated plunger bears against the tail end thereof;
wherein the turned-in axially-elongated flanges of every clip track along the parallel opposed edges of both arms, whereby the arms of the tubular conduit and flanges of the lead clip cooperate to prevent relative spinning and binding among the clips when the endoscope is flexed in a tight curvature and thereby present the lead clip in proper orientation for the flanges thereof to engage the wedge-shaped flared ends.

5. The combination of claim 4, wherein:
the elongated plunger rod is configured with a stroke for pushing the line of clips through the lumen of the tubular conduit at an incrementally indexed or metered rate of one clip at a time.

6. The combination of claim 4, wherein:
the one and the other pair of turned-in axially-elongated flanges that flank the one and other jaw of each clip are elongated between a rear end and front end, and clasp and engage the arms over a length between the rear end and front end while progressing toward the wedge-shaped flared ends whereby to better promote orientation relative the clips and arms as well as prevent binding and/or relative spinning therebetween.

7. The combination of claim 2, further comprising:
an elongated outer tubular conduit in which said elongated tubular conduit is sleeved inside thereof, whereby said elongated tubular conduit serves as an elongated inner tubular conduit relative thereto;
said elongated outer tubular conduit terminating in a dispensing end;
said elongated inner tubular conduit being sized to closely fit in the elongated outer tubular conduit with the terminal end of the elongated inner tubular conduit proximate the dispensing end of the elongated outer tubular conduit.

8. The combination of claim 7, wherein:
the wedge-shaped flared ends are disposed past the dispensing end of the elongated outer tubular conduit while the turned-in axially-elongated flanges that flank the one and other jaw of the lead clip are engaged therewith.

9. The combination of claim 8, wherein:
the one and the other pair of turned-in axially-elongated flanges that flank the one and other jaw of each clip are furthermore configured for partly clasping the arms where the edges thereof are parallel while tracking therealong or the wedge-shaped flared ends while engaged therewith.

10. The combination of claim 8, wherein:
the elongated plunger has an extension stroke comprising plural increments corresponding to the plurality of clips, one incremental extension stroke per clip; and
the inner tubular conduit has an extension and retraction stroke per clip over a length, said length of the extension and retraction stroke being independent of the incremental stroke of the elongated plunger, but the extension stroke is synchronized with of the elongated inner tubular conduit and the incremental extension stroke of the elongated plunger are linked for synchronization.

11. The combination of claim 2, wherein:
the pair of opposed slender arms that ultimately terminate in the wedge-shaped flared ends are affixed to an inside wall of the elongated inner tubular conduit and project out away from the terminal end of the elongated inner tubular conduit.

12. The combination of claim 10, wherein:
the wedge-shaped flared ends are extended past the dispensing end of the elongated outer tubular conduit with the extension stroke of the elongated inner tubular conduit and before the turned-in axially-elongated flanges that flank the one and other jaw of the lead clip are engaged therewith; and
after the turned-in axially-elongated flanges that flank the one and other jaw of the lead clip have passed their engagement with the flared apart edges of the wedge-shaped flared ends, coinciding about when the jaws of the lead clip are free to snap shut and ‘bite’ into a target anatomical structure, the wedge-shaped flared ends retract back inside and behind the plane of the dispensing end of the elongated outer tubular conduit with the retraction stroke of the elongated inner tubular conduit.

13. The combination of claim 12, wherein:
said dispensing end of said outer tubular conduit lies in a plane;
said terminal end of the elongated inner tubular conduit always remains inside and behind the plane of the dispensing end of the elongated outer tubular conduit with both the extension and retraction strokes of the elongated inner tubular conduit.

14. The combination of claim 13, further comprising:
a manual actuator for the elongated plunger and inner tubular conduit which has a stroke corresponding to the length of one incremental stroke of the elongated plunger.

15. The combination of claim 14, wherein:
each elongated clip is characterized by a length; and
the length of one incremental stroke of the elongated plunger corresponds to the length each elongated clip.

16. A combination endoscope, endoscopic multi-clip dispensing apparatus and a plurality of clips therefor, comprising:
an elongated flexible endoscope extending between an operator end and a probe end;
said elongated endoscope comprising at least an internal optics channel with a lens proximate the probe end, a source of illumination proximate the probe end, control wires that control the flexure of the endoscope, and an internal working channel having a forward opening in the probe end and a rearward opening proximate the operator end;
an flexible axially-elongated tubular conduit having an inside wall defining an axial lumen, terminating in a terminal end;
a pair of opposed axially-elongated slender arms disposed inside the lumen of the axially-elongated tubular conduit and affixed to the inside wall of the axially-elongated tubular conduit, which arms which project past the terminal end thereof and away therefrom;
each arm ultimately terminating in a wedge-shaped flared end past the terminal end of the axially-elongated tubular conduit;
each arm having a pair of angularly-spaced axially-elongated edges which are parallel to one another except for the wedge-shaped flared end, wherein said edges for said arm flare apart at the wedge-shaped flared end;
a plurality of clips, each having a C-shape characterized by a pair of elongated jaws mated together at a hinge end, wherein the jaws have tip ends forming a head end for the clip and hinge end forms a tail end for the clip when said plurality of clips are loaded lumen of the axially-elongated tubular conduit in head to tail fashion between a lead clip and last clip;
each clip having one and another pair of turned-in axially-elongated spaced flanges, one pair flanking one jaw and the other pair flanking the other jaw, each turned-in axially-elongated flange extending between a head end and a tail end; and
a flexible axially-elongated plunger sleeved inside the lumen of the axially-elongated tubular conduit for pushing the lead clip up to and past the wedge-shaped flared ends which are affixed relative thereto;
wherein the turned-in axially-elongated flanges of the plurality of clips loaded into the lumen of the axially-elongated tubular conduit in head to tail fashion are configured to clasp the arms about the edges thereof to prevent binding and relative spinning among the clips when the axially-elongated conduit is flexed in a tight curvature and thereby present the lead clip in proper orientation for the flanges thereof to engage the wedge-shaped flared ends; and
wherein said axially-elongated tubular conduit is threaded through the working channel of the endoscope and operated thereafter from outside a patient to dispense clips inside the patient's anatomy.

17. The combination of claim 16, wherein:
said plurality of clips are configured for, when loaded into the lumen of the axially-elongated tubular conduit in head to tail fashion, allowing relative articulation therebetween;

18. The combination of claim 16, wherein:
the one and the other pair of turned-in axially-elongated flanges that flank the one and other jaw of each clip are elongated between a rear end and front end, and clasp and engage the arms over a length between the rear end and front end while progressing toward the wedge-shaped flared ends whereby to better promote orientation relative the clips and arms as well as prevent binding and/or relative spinning therebetween.
